SEQUENCE LISTING (32 sequences)

SEQ ID NO: 1 - DNA, 4795 bp, Mus musculus
  source 1..4795: mol_type = other DNA
gagcattcactctagcgagcgagcggcgtgtacagccggctccctgggctcctggagtcccgcttgctccaagcgcactccagcagtctctttctgctcttgcccggctcgacggcgacatgggtgtccccgcggtcccagaggccagcagcccgcgctggggaaccctgctccttgctattttcctggctgcatccagaggtctggtagcagccttcaaggtcaccactccatattctctctatgtgtgtcccgagggacagaatgccaccctcacctgcaggattctgggccccgtgtccaaagggcacgatgtgaccatctacaagacgtggtacctcagctcacgaggcgaggtccagatgtgcaaagaacaccggcccatacgcaacttcacattgcagcaccttcagcaccacggaagccacctgaaagccaacgccagccatgaccagccccagaagcatgggctagagctagcttctgaccaccacggtaacttctctatcaccctgcgcaatgtgaccccaagggacagcggcctctactgctgtctagtgatagaattaaaaaaccaccacccagaacaacggttctacgggtccatggagctacaggtacaggcaggcaaaggctcggggtccacatgcatggcgtctaatgagcaggacagtgacagcatcacggctgcggccctggccaccggcgcctgcatcgtgggaatcctctgcctcccccttatcctgctgctggtctataagcagagacaggtggcctctcaccgccgtgcccaggagttggtgaggatggacagcagcaacacccaaggaatcgaaaacccaggcttcgagaccactccacccttccaggggatgcctgaggccaagaccaggccgccactgtcctatgtggcccagcggcaaccttcggagtcaggacggtacctgctctctgaccccagcacacctctgtcgcctccaggccctggggacgtctttttcccatccctagatccagtccctgactcccctaactctgaagccatctaaaccagctggggaaccatgaaccatggtacctgggtcagggatatgtgcacttgatctatggctggcccttggacagtcttttaggcactgactccagcttccttgctcctgctctgagcctagactctgcttttacaagatgcacagaccctcccctatctctttcagacgctacttggggggcagggagaagatgttggattgctcatggctgttctcaagatcttgggatgctgagttctccctagagacttgacttcgacagccacagatgtcagatgacctgcatcctatgaacgtccggcttggcaagagcctttcttcatggaaaccagtagcccggaggggatgaggtaggcaccttgccaccctcccgggagagagacacaagatgtgagagactcctgctcactgtgggggtgtggctggcctgcttgtttgcctgaggatgctcctctgttggactgactctatccccctggattctggagcttggctggcctatgtcccaccagaggagcatctcagcagccttccaccagcaacctgagggcctgccagcttcgtggctctgggctctcattacctgtatggccgtccacagagctcagtggccagaggctttgaaacaggaagtacatgtcaggttcaggaaccactgtgagctcattagtgtcttgagcaatgtgaggcctggaccagtggacacggagggagggtggcgagaggatgatggggatgatgaggggaacacgctcccttcctgtccttgtcatccaccactaccactattcagtgtggagcagtggcaaaggtgaccgacctccacaatgtcctagtgatgctggaccatttctaagtgtgaaagagatgctattaaaaacagtatgtggcaatggctgccaacagctgagtggactggaggcactggctttaaggccctggaggtgcagggcccggtatggggatagggatgggagtttcagtgagggcctagggatcactccgcttctgaccactcttcttctgagcctcacctcagggtgaccttcaggcacacagaagagcttgcccctggtccgatactactcttggctctcatctccagggtttggcatgacctgggcacacagggggagtcttcagaaaggattttaaagcatgaaaagaaagggtagttcttgtgaggtagggatgggcagctgatgtttgagagtgaggagggatacggctgggcagatcactctccagtctctagagggaaagtagctctaagtctgggagagcagcagcccagtggtaccatatgtcttcttgcagcttccactggctgggctgaactgggcatgggtaggaaagctcctgttctgggcctgcagccagggagaaccccattcattccctgaggacagatgggtggggagagaagagagagtttcaggccgggaagcagcaataagctatctgctggggacccagacaagttgtctgatgaggtccaagatgtgggatgccagttatacctggggcttggggatccttagaggctttgtatcatcatcataggagtgtcggggtggccagggcatcaaagccatgacccctgttttatcctcagggtccactcttctgcaccatccattgctctagatctatgcagttactatagacagaatgtgttgttctgtttggctttggggataatggcctggcgaactgccagctgttcagtggcagggctgtgaggccagtcaaagactagaacccacagaccagctgaacgatgagtatagcctgtcccctgggggagcctgacctgtctccagccctaagcttcagacctcaccactcagatgacttctaagaatttgcctgtggggacccctgcatggctgcagctccgtggaaaggagaggaggcccccagcagaagaaccactcgcttcctgcccagcttcctcctgtagggctctaagtctcttcttcttgggaccctgcaagcaaaggcatgtcagcttggtggtttcctgttttgggtgaagttttgtgtggtccgggttctgtctacatccatgaacttggggtgctaccaccttgctgctgctgtagagacagctgcaggatcttagggtggaaaatggaggtgccctgaggtgctagcccttggggcaaaagatggggtggcaatgagacacagtggggaactgagttccccaagaggagggaggagccctgtagcctcaagggccatattgggttcctggtaccagcaaaagcctagagagcgaagtctgtattttgaggaggtaattgatccttacggaatccatcagaaatttggagcgggtgctttatctatctctggagggtctctacctatctccgatgaagctctccctgggcctgggatgggagaaaccaggaggaaaggtgtctgataaagcaggggcttcttgacaagccaaagggccactggtagctgttgtggaccgagctgaccctgctgaagtattgtagtgtgccttggaccaacttctcaaaagagcaaccccggggctaccctacttctgccaggaagaggcggagaaggggctgagaggcctggaaggggctagctccttctttgagaactgctccccggaggacttggaggaggcggctaggctacgggctgctgagggccctttgtctttcctaacctgggcactgttaggatgctccctcctggaaaaggctttcctgggtgtgagctagagcagtgtccatgccagcgctgaacctgccatggtgggagctgaactaaaaatttctcagggaactaaaataggcaaaagaggaactgggggaggagggtgccaggcaggatggggggaagggagggcagtgcaaaagtctcttgaaacacagacagcccagctgagtgccagtcccagatcacagagaatacggctcatctggctcatgttctgcatgcttgctgctttaccctggcactttccttctccaccatgagtgcgagtcctgggagtcctgggagggtgaggattaatgccagcttggtgagcagatagctgacagagtccttgggtaactggcttgaaccaggacctcaggattccactctggggatctagctttgtctgggccagtgaagatctctataatggcattattgccaggggataaacatttcactgggttctgatctgttgggtgtggcttcctggaaaatatggtgagaggaattctgctaaggatacagttgataagaaagttctgagattgattagtaatgcctgccttggactcaggaagggaagtggcagtatgaatgccatgtcttaatcattttggttaaaatatgcttcccaaaagatttccacgtgtgttcttgtttatttgacatctgtctccatatcagtcttgaaagcctttctgtgtgtatatatatgatgtttgcgtgtatatatgtttttgtgtgtgcatatggaagtcagaaatcactgggtgtcttcctccattcctttgcaatgtatgttttttttttttttacgatttatttactatatgaatgttttgcctgaatacatgcataggtgtcacgtacatgcctgctggaacgcttggaactggagttacaggtggctatgagctacagtgtgagcactgggaatcaaacctgggtcttctgcaagagcaacaaattaaaagtcagctcttaactacttgagctatttttccaactcc

SEQ ID NO: 2 - DNA, 4774 bp, Homo sapiens
  source 1..4774: mol_type = other DNA
gggggcgggtgcctggagcacggcgctggggccgcccgcagcgctcactcgctcgcactcagtcgcgggaggcttccccgcgccggccgcgtcccgcccgctccccggcaccagaagttcctctgcgcgtccgacggcgacatgggcgtccccacggccctggaggccggcagctggcgctggggatccctgctcttcgctctcttcctggctgcgtccctaggtccggtggcagccttcaaggtcgccacgccgtattccctgtatgtctgtcccgaggggcagaacgtcaccctcacctgcaggctcttgggccctgtggacaaagggcacgatgtgaccttctacaagacgtggtaccgcagctcgaggggcgaggtgcagacctgctcagagcgccggcccatccgcaacctcacgttccaggaccttcacctgcaccatggaggccaccaggctgccaacaccagccacgacctggctcagcgccacgggctggagtcggcctccgaccaccatggcaacttctccatcaccatgcgcaacctgaccctgctggatagcggcctctactgctgcctggtggtggagatcaggcaccaccactcggagcacagggtccatggtgccatgagctgtcaggtgcagacaggcaaagatgcaccatccaactgtgtggtgtacccatcctcctcccaggatagtgaaaacatcacggctgcagccctggctacgggtgcctgcatcgtaggaatcctctgcctccccctcatcctgctcctggtctacaagcaaaggcaggcagcctccaaccgccgtgcccaggagctggtgcggatggacagcaacattcaagggattgaaaaccccggctttgaagcctcaccacctgcccaggggatacccgaggccaaagtcaggcaccccctgtcctatgtggcccagcggcagccttctgagtctgggcggcatctgctttcggagcccagcacccccctgtctcctccaggccccggagacgtcttcttcccatccctggaccctgtccctgactctccaaactttgaggtcatctagcccagctgggggacagtgggctgttgtggctgggtctggggcaggtgcatttgagccagggctggctctgtgagtggcctccttggcctcggccctggttccctccctcctgctctgggctcagatactgtgacatcccagaagcccagcccctcaacccctctggatgctacatggggatgctggacggctcagcccctgttccaaggattttggggtgctgagattctcccctagagacctgaaattcaccagctacagatgccaaatgacttacatcttaagaagtctcagaacgtccagcccttcagcagctctcgttctgagacatgagccttgggatgtggcagcatcagtgggacaagatggacactgggccaccctcccaggcaccagacacagggcacggtggagagacttctcccccgtggccgccttggctcccccgttttgcccgaggctgctcttctgtcagacttcctctttgtaccacagtggctctggggccaggcctgcctgcccactggccatcgccaccttccccagctgcctcctaccagcagtttctctgaagatctgtcaacaggttaagtcaatctggggcttccactgcctgcattccagtccccagagcttggtggtcccgaaacgggaagtacatattggggcatggtggcctccgtgagcaaatggtgtcttgggcaatctgaggccaggacagatgttgccccacccactggagatggtgctgagggaggtgggtggggccttctgggaaggtgagtggagaggggcacctgccccccgccctccccatcccctactcccactgctcagcgcgggccattgcaagggtgccacacaatgtcttgtccaccctgggacacttctgagtatgaagcgggatgctattaaaaactacatggggaaacaggtgcaaaccctggagatggattgtaagagccagtttaaatctgcactctgctgctcctcccccacccccaccttccactccatacaatctgggcctggtggagtcttcgcttcagagccattcggccaggtgcgggtgatgttcccatctcctgcttgtgggcatgccctggctttgtttttatacacataggcaaggtgagtcctctgtggaattgtgattgaaggattttaaagcaggggaggagagtagggggcatctctgtacactctgggggtaaaacagggaaggcagtgcctgagcatggggacaggtgaggtggggctgggcagaccccctgtagcgtttagcaggatgggggccccaggtactgtggagagcatagtccagcctgggcatttgtctcctagcagcctacactggctctgctgagctgggcctgggtgctgaaagccaggatttggggctaggcgggaagatgttcgcccaattgcttggggggttggggggatggaaaaggggagcacctctaggctgcctggcagcagtgagccctgggcctgtggctacagccagggaaccccacctggacacatggccctgcttctaagccccccagttaggcccaaaggaatggtccactgagggcctcctgctctgcctgggctgggccaggggctttgaggagagggtaaacataggcccggagatggggctgacacctcgagtggccagaatatgcccaaaccccggcttctcccttgtccctaggcagaggggggtcccttcttttgttccctctggtcaccacaatgcttgatgccagctgccataggaagagggtgctggctggccatggtggcacacacctgtcctcccagcactttgcagggctgaggtggaaggaccgcttaagcccaggtgttcaaggctgctgtgagctgtgttcgagccactacactccagcctggggacggagcaaaactttgcctcaaaacaaattttaaaaagaaagaaagaaggaaagagggtatgtttttcacaattcatgggggcctgcatggcaggagtggggacaggacacctgctgttcctggagtcgaaggacaagcccacagcccagattccggttctcccaactcaggaagagcatgccctgccctctggggaggctggcctggccccagccctcagctgctgaccttgaggcagagacaacttctaagaatttggctgccagaccccaggcctggctgctgctgtgtggagagggaggcggcccgcagcagaacagccaccgcacttcctcctcagcttcctctggtgcggccctgccctctcttctctggacccttttacaactgaacgcatctgggcttcgtggtttcctgttttcagcgaaatttactctgagctcccagttccatcttcatccatggccacaggccctgcctacaacgcactagggacgtccctccctgctgctgctggggaggggcaggctgctggagccgccctctgagttgcccgggatggtagtgcctctgatgccagccctggtggctgtgggctggggtgcatgggagagctgggtgcgagaacatggcgcctccagggggcgggaggagcactaggggctggggcaggaggctcctggagcgctggattcctggcacagtctgaggccctgagagggaaatccatgcttttaagaactaattcattgttaggagatcaatcaggaattaggggccatcttacctatctcctgacattcacagtttaatagagacttcctgcctttattccctcccagggagaggctgaaggaatggaattgaaagcaccatttggagggttttgctgacacagcggggactgctcagcactccctaaaaacacaccatggaggccactggtgactgctggtgggcaggctggccctgcctgggggagtccgtggcgatgggcgctggggtggaggtgcaggagccccaggacctgcttttcaaaagacttctgcctgaccagagctcccactacatgcagtggcccagggcagaggggctgatacatggcctttttcagggggtgctcctcgcggggtggacttgggagtgtgcagtgggacagggggctgcaggggtcctgccaccaccgagcaccaacttggcccctggggtcctgcctcatgaatgaggccttccccagggctggcctgactgtgctgggggctgggttaacgttttctcagggaaccacaatgcacgaaagaggaactggggttgctaaccaggatgctgggaacaaaggcctcttgaagcccagccacagcccagctgagcatgaggcccagcccatagacggcacaggccacctggcccattccctgggcattccctgctttgcattgctgcttctcttcaccccatggaggctatgtcaccctaactatcctggaatgtgttgagagggattctgaatgatcaatatagcttggtgagacagtgccgagatagatagccatgtctgccttgggcacgggagagggaagtggcagcatgcatgctgtttcttggccttttctgttagaatacttggtgctttccaacacactttcacatgtgttgtaacttgtttgatccacccccttccctgaaaatcctgggaggttttattgctgccatttaacacagagggcaatagaggttctgaaaggtctgtgtcttgtcaaaacaagtaaacggtggaactacgactaaa

SEQ ID NO: 3 - DNA, 25 bp, synthetic construct
  source 1..25: mol_type = other DNA
gggcacgatgtgaccttctacaaga

SEQ ID NO: 4 - DNA, 25 bp, synthetic construct
  source 1..25: mol_type = other DNA
cagatgccaaatgacttacatctta

SEQ ID NO: 5 - DNA, 25 bp, synthetic construct
  source 1..25: mol_type = other DNA
gagatggattgtaagagccagttta

SEQ ID NO: 6 - DNA, 25 bp, synthetic construct
  source 1..25: mol_type = other DNA
gggctttgaggagagggtaaacata

SEQ ID NO: 7 - DNA, 25 bp, synthetic construct
  source 1..25: mol_type = other DNA
cctatctcctgacattcacagttta

SEQ ID NO: 8 - DNA, 25 bp, synthetic construct
  source 1..25: mol_type = other DNA
cagtttaatagagacttcctgcctt

SEQ ID NO: 9 - DNA, 25 bp, synthetic construct
  source 1..25: mol_type = other DNA
cagggagaggctgaaggaatggaat

SEQ ID NO: 10 - DNA, 25 bp, synthetic construct
  source 1..25: mol_type = other DNA
ggattgtgttgagagggattctgaa

SEQ ID NO: 11 - DNA, 25 bp, synthetic construct
  source 1..25: mol_type = other DNA
gagagggattctgaatgatcaatat

SEQ ID NO: 12 - DNA, 25 bp, synthetic construct
  source 1..25: mol_type = other DNA
cacagagggcaatagaggttctgaa

SEQ ID NO: 13 - DNA, 25 bp, synthetic construct
  source 1..25: mol_type = other DNA
cagatgccaaatgacttacatctta

SEQ ID NO: 14 - DNA, 25 bp, synthetic construct
  source 1..25: mol_type = other DNA
gagatggattgtaagagccagttta

SEQ ID NO: 15 - DNA, 25 bp, synthetic construct
  source 1..25: mol_type = other DNA
ggtgagtcctctgtggaattgtgat

SEQ ID NO: 16 - DNA, 25 bp, synthetic construct
  source 1..25: mol_type = other DNA
gggctttgaggagagggtaaacata

SEQ ID NO: 17 - DNA, 25 bp, synthetic construct
  source 1..25: mol_type = other DNA
cctatctcctgacattcacagttta

SEQ ID NO: 18 - DNA, 25 bp, synthetic construct
  source 1..25: mol_type = other DNA
cagtttaatagagacttcctgcctt

SEQ ID NO: 19 - DNA, 25 bp, synthetic construct
  source 1..25: mol_type = other DNA
cagggagaggctgaaggaatggaat

SEQ ID NO: 20 - DNA, 25 bp, synthetic construct
  source 1..25: mol_type = other DNA
ggaatgtgttgagagggattctgaa

SEQ ID NO: 21 - DNA, 25 bp, synthetic construct
  source 1..25: mol_type = other DNA
gagagggattctgaatgatcaatat

SEQ ID NO: 22 - DNA, 25 bp, synthetic construct
  source 1..25: mol_type = other DNA
cacagagggcaatagaggttctgaa

SEQ ID NO: 23 - DNA, 25 bp, synthetic construct
  source 1..25: mol_type = other DNA
acaaagggcacgatgtgaccttcta

SEQ ID NO: 24 - DNA, 25 bp, synthetic construct
  source 1..25: mol_type = other DNA
gggcacgatgtgaccttctacaaga

SEQ ID NO: 25 - DNA, 25 bp, synthetic construct
  source 1..25: mol_type = other DNA
gaccaccatggcaacttctccatca

SEQ ID NO: 26 - DNA, 25 bp, synthetic construct
  source 1..25: mol_type = other DNA
cagacaggcaaagatgcaccatcca

SEQ ID NO: 27 - DNA, 25 bp, synthetic construct
  source 1..25: mol_type = other DNA
ggcaaagatgcaccatccaactgtg

SEQ ID NO: 28 - DNA, 25 bp, synthetic construct
  source 1..25: mol_type = other DNA
ccatccaactgtgtggtgtacccat

SEQ ID NO: 29 - DNA, 25 bp, synthetic construct
  source 1..25: mol_type = other DNA
ggatggacagcaacattcaagggat

SEQ ID NO: 30 - DNA, 25 bp, synthetic construct
  source 1..25: mol_type = other DNA
gacagcaacattcaagggattgaaa

SEQ ID NO: 31 - DNA, 25 bp, synthetic construct
  source 1..25: mol_type = other DNA
ccctgtccctgactctccaaacttt

SEQ ID NO: 32 - DNA, 25 bp, synthetic construct
  source 1..25: mol_type = other DNA
cctgactctccaaactttgaggtca

(19) United States
(12) Patent Application Publication
(10) Pub. No.: US 2025/0295806 A1
(43) Pub. Date: Sep. 25, 2025

(54) IMMUNOSTIMULATORY NANOPARTICLE

(71) Applicants: CASE WESTERN RESERVE UNIVERSITY, Cleveland, OH (US); Cleveland Clinic Foundation, Cleveland, OH (US)

(72) Inventors: Efsthatios Karathanasis, Cleveland, OH (US); Taylor Moon, Cleveland, OH (US); Morgan Lorkowski, Cleveland, OH (US); Li Lily Wang, Cleveland, OH (US)

(21) Appl. No.: 18/862,998

(22) PCT Filed: May 5, 2023
(86) PCT No.: PCT/US2023/021141

§ 371 Date: Nov. 5, 2024

Related U.S. Application Data
(60) Provisional application No. 63/338,543, filed on May 5, 2022.

(51) Int. Cl. A61K 47/69; A61K 9/51; A61K 31/7088; A61K 47/55; A61P 35/00; C12N 15/113

(52) U.S. Cl. CPC A61K 47/6925; A61K 9/5123; A61K 31/7088; A61K 47/551; A61P 35/00; C12N 15/1138; C12N 2310/14

(57) ABSTRACT

An immunostimulatory nanoparticle comprising a biocompatible lipid shell that defines an outer surface of the nanoparticle and a core, which is loaded with a Toll-like Receptor 9 (TLR9) agonist and a nucleic acid inhibitor of V domain Immunoglobulin Suppressor of T cell activation (VISTA), and optionally a plurality of targeting moieties linked to the outer surface, wherein the optional targeting moieties are configured to direct the nanoparticle to tumor-resident myeloid cells in a tumor microenvironment upon administration of the nanoparticle to a subject with cancer.

### RELATED APPLICATION

This application claims priority from U.S. Provisional Application No. 63/338,543, filed May 5, 2022, the subject matter of which is incorporated herein by reference in its entirety.

### GOVERNMENT FUNDING

This invention was made with government support under CA164225 and CA253627 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on May 4, 2023, is named CWR-031408WO ORD st.26 and is 38,837 bytes in size.

### BACKGROUND

Novel cancer therapies targeting treatment resistance, metastasis and disease relapse while minimizing side effects and toxicity are urgently needed for the aggressive forms of cancer. Immunotherapy encompasses an inherent premise to treat aggressive and metastatic cancer by eliciting T cell-mediated killing of tumor cells. Effective antitumor immunity depends on overcoming the profound immunosuppression within the tumor microenvironment (TME). The TME is typically enriched with dysfunctional and immunosuppressive myeloid cells, including macrophages, dendritic cells (DCs), and myeloid-derived suppressor cells (MDSCs).

Notably to date, most cancer patients have had suboptimal responses to immunotherapies largely due to the local immunosuppressive barrier in tumors. This non-inflamed “cold” and unreceptive microenvironment inhibits activated systemic lymphocytes from effectively trafficking to tumor sites, thereby shielding developing tumors from systemic immuno-surveillance.

Generating pro-inflammatory innate immunity at the tumor site is perhaps the single most pivotal factor that drives the success of an immunotherapy. Inflamed “hot” tumors that are rich in stimulatory myeloid cells are receptive to immunotherapeutic intervention because these cells activate local immunity and recruit systemic immunity, thereby orchestrating clearance from within the tumor itself. Such approach can facilitate an antitumor immune response leading to tumor rejection and prevention of disease relapse.

### SUMMARY

Embodiments described herein relate to immunostimulatory nanoparticles and their use in treating cancer. The nanoparticles include a biocompatible lipid shell that defines an outer surface of the nanoparticle and a core, which is loaded with a Toll-like Receptor 9 (TLR9) agonist and a nucleic acid inhibitor of V domain Immunoglobulin Suppressor of T cell activation (VISTA). The immunostimulatory nanoparticles optionally include a plurality of targeting moieties linked to the shell and extending from the outer surface. The optional targeting moieties are configured to direct the nanoparticle to tumor-resident myeloid cells in a tumor microenvironment upon administration of the nanoparticle to a subject with cancer.

In some embodiments, the TLR9 agonist is a CpG oligonucleotide, such as a CpG dinucleotide, and the nucleic acid inhibitor of VISTA includes an RNAi construct that inhibits VISTA expression, such as an siRNA targeting VISTA expression.

In some embodiments, the ratio of siRNA/CpG loaded into the core is about 10:1 to about 1:10, preferably about 5:1 to about 1:1.

In some embodiments, the lipid shell is configured to shield the TLR9 agonist and the nucleic acid inhibitor of VISTA from degradation upon administration to the subject and release the TLR9 agonist and the nucleic acid inhibitor of VISTA upon internalization of the nanoparticle by a myeloid cell. The lipid shell of the nanoparticle can include at least one ionizable cationic lipid. The lipid shell can also include a phospholipid that is optionally functionalized with polyethylene glycol (PEG), cholesterol, and other lipids. For example, the lipid shell can include about 42 mol % to about 50 mol % of an ionizable cationic lipid, about 38 mol % to about 40 mol % cholesterol, and about 11 mol % to about 14 mol % of a phospholipid.

In some embodiments, the nanoparticle has a diameter of about 20 nm to about 1 μm, preferably about 30 nm to about 100 nm, or more preferably about 30 nm to about 60 nm.

In other embodiments, the targeting moiety can include a ligand that specifically binds to folate receptor beta on myeloid cells. For example, the ligand can include folate that is linked to shell of the nanoparticle with a PEG linker.

Other embodiments described herein relate to an immunotherapy composition that includes a plurality of immunotherapy nanoparticles as described. The composition can further include a pharmaceutically acceptable carrier.

Still other embodiments relate to a method of treating cancer in a subject in need thereof. The method includes administering to the subject a therapeutically effective amount of the composition described herein.

In some embodiments, the composition is administered by systemic administration, intra-tumoral, peri-tumoral, and directly into tumor draining lymph node(s).

In some embodiments, the therapeutically effective amount is an amount effective to reprogram and activate local tumor-resident myeloid cells into T cell-stimulatory cells in the subject and promote cytotoxic CD8+ T-cell mediated killing of tumor cells.

In other embodiments, the therapeutically effective amount is an amount effective to reduce tumor burden in a subject.

In some embodiments, one or more additional cancer therapies can be administered to the subject. The one or more additional cancer therapies include ablation therapy, radiation therapy, surgery, chemotherapy, and an immunotherapy. The immunotherapy can include one or more therapeutic antibodies and/or one or more immune checkpoint inhibitors.

In some embodiments, the cancer can include a low immunogenic cancer or cancers characterized by immunological escape. In certain embodiments, the cancer treated can include a metastatic cancer. In certain embodiments, the cancer is selected from the group consisting of melanoma, colon cancer, and ovarian cancer. In some embodiments, the cancer treated in the subject is metastic melanoma.

### BRIEF DESCRIPTION OF THE FIGURES

FIGS. 1(A-C) is schematic of the dual action immunostimulatory nanoparticle (immuno-NP) platform showing (A) the tumor immune microenvironment with dysfunctional myeloid cells, (B) the immuno-NP loaded with the synergistic dual cargo of the TLR9 agonist CpG and the VISTA siRNA, and (C) the targeting scheme to direct the immuno-NP specifically to dysfunctional myeloid cells and the proficient presentation of the two cargoes to the appropriate intracellular locations.

FIGS. 2(A-E) illustrate plots and graphs showing (A) DLS measurements were used to assess the effect of lipid composition on the size of dual-NP. The immuno-NP variants were sonicated for 4 min. (B) DLS used to analyze the effect of sonication time on the size of immuno-NP. The lipid composition was based on a 7 mol % total PEG content. (C) The zeta potential of immuno-NP during VISTA siRNA and CpG loading and after cleaning. (D) Loading efficiency of siRNA and CpG into immuno-NP. (E) NP stability in terms of hydrodynamic size as measured by DLS.

FIGS. 3(A-C) illustrate graphs showing flow cytometry analysis of the cellular uptake of immuno-NPs by (A) DCs, (B) macrophages, and (C) gMDSC in the B16F10 tumor and blood 24 h after i.t. administration (N=5 mice per group). Mean±standard deviation are plotted with statistics by a 2-tailed t-test (ns=not significant, ***P<0.001, ****P<0.0001).

FIGS. 4(A-D) illustrate a schematic and plot showing the global effect of the immuno-NP treatment. (A) A mouse model with two simultaneous B16F10 tumors was developed. A secondary tumor (100k cells) was inoculated 3 days after the primary tumor (500k cells). The immuno-NP was administered i.t. at a dose of 15 μg VISTA siRNA and 6 μg CpG on days 12 and 19 after tumor inoculation of B16F10 melanoma cells. Tumors were ˜100 mm³ at the time of treatment. (B) Survival rate. The volume of the (C) primary and (D) secondary tumor was monitored using caliper measurements (n=5 mice per group).

FIGS. 5(A-G) illustrate plots and an image showing efficacy of the immuno-NP treatment. (A) The immuno-NP was administered i.t. at a dose of 10 μg VISTA siRNA and 4 μg CpG on days 12, 19 and 26 after tumor inoculation of B16F10 melanoma cells. Tumors were about 100 mm³ at the time of treatment. The control treatments were administered at the same schedule and dose. The volume of the primary tumor was monitored using caliper measurements (n=10 for the immuno-NP group; n=5 mice for all other groups). (B) Tumor growth of the animals in the immuno-NP group (n=10). (C) Survival rate. (D) Body weights of the mice treated with immuno-NP. The mice with complete response were re-challenged in the opposite flank with 100,000 tumor cells and the tumor volume (E) and survival (F) was monitored for 2 months (n=5 per group). (G) To simulate metastatic recurrence, the same animals were re-challenged again 2 months later via intravenous injection of 200,000 tumor cells. At the near-terminal point of the untreated control mice (day 24 after intravenous re-challenge), all mice were euthanized, lungs were excised and photographed (n=5 per group).

FIG. 6 illustrates plots showing bone marrow VISTA expression from WT or VISTA KO mice. After depletion of granulocytes, macrophages, B, T, and NK cells, cells were cultured with GM-CSF and IL6 in the presence of the VISTA-targeting siRNA in its free form or as immuno-NP at 25 nM siRNA concentration. VISTA expression on DC and MDSCs subsets were examined by flow cytometry at 48 and 72 h after treatment.

FIGS. 7(A-D) illustrate graphs showing that the synergistic action of immuno-NP promotes pro-inflammatory activity. Mice bearing B16F10 tumors were treated intratumorally (i.t.) with two doses of immuno-NP in subsequent days. Each dose (20 μL) contained 10 μg VISTA siRNA and 4 μg CpG. Treatment was administered when tumors were about 100 mm³. Flow cytometry analysis shows (A) the VISTA⁺ M1 TAM population, the mMDSC population and the VISTA+mMDSCs. ELISA measurement for IL12p70 (left panel), IFNγ (middle panel) and IL2 (right panel). N=5 mice per group (data are plotted as mean±standard error; t-test analysis).

FIGS. 8(A-E) illustrate graphs and plots showing safety studies in mice. (a) Immuno-NP was injected i.t. in mice with B16 melanoma tumors at a dose of 10 μg VISTA siRNA and 4 μg CpG. Control conditions included PBS (i.t.) and soluble VISTA mAb and CpG (i.v.). Blood was collected at various time points. Cell count for (A) leukocytes and lymphocytes, and (B) neutrophils and monocytes. ELISA measurement for (C) IL12p70, (D) ALT and (E) AST. N-5 mice per group. Data are plotted as mean±standard error; 2-way ANOVA with Tukey's/Sidak's post-test (n.s. not significant, ****P<0.0001).

FIGS. 9(A-D) illustrate graphs showing immuno-NPs targeted to myeloid cells in the tumor microenvironment (TME). (A) Flow cytometry analysis of myeloid cells in the primary tumor using the 4T1 mouse model. Tumor-resident VISTA⁺ and folate⁺ myeloid, (B) tumor-resident VISTA⁺ and folate⁺ M2 TAM, G-MDSC and M-MDSC, and (C) VISTA⁺ and folate⁺ M2 TAM, G-MDSC and M-MDSC in healthy tissues (lungs). (D) Cellular uptake of FR-targeted and untargeted immuno-NPs by myeloid cells in the B16F10 tumor and blood 24 h after i.t. administration (N=5 mice per group). Mean±standard deviation are plotted with statistics by a 2-tailed t-test (ns=not significant, *P<0.05, **P<0.01, ***P<0.001, ****P<0.0001).

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the application pertains. Commonly understood definitions of molecular biology terms can be found in, for example, Rieger et al., Glossary of Genetics: Classical and Molecular, 5th Edition, Springer-Verlag: New York, 1991, and Lewin, Genes V, Oxford University Press: New York, 1994.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, and other references mentioned herein are incorporated by reference in their entirety.

As used in the description of the invention and the appended claims, the singular forms “a,” “an,” and “the” are intended to include the plural forms as well, unless the context clearly indicates otherwise. In addition, the recitations of numerical ranges by endpoints include all numbers subsumed within that range (e.g., 1 to 5 includes 1, 1.5, 2, 2.75, 3, 3.80, 4, 5, etc.).

The terms “comprise,” “comprising,” “include,” “including,” “have,” and “having” are used in the inclusive, open sense, meaning that additional elements may be included. The terms “such as”, “e.g.,”, as used herein are non-limiting and are for illustrative purposes only. “Including” and “including but not limited to” are used interchangeably.

The term “or” as used herein should be understood to mean “and/or”, unless the context clearly indicates otherwise.

The terms “cancer” or “tumor” refer to any neoplastic growth in a subject, including an initial tumor and any metastases. The cancer can be of the liquid or solid tumor type. Liquid tumors include tumors of hematological origin, including, e.g., myelomas (e.g., multiple myeloma), leukemias (e.g., Waldenstrom's syndrome, chronic lymphocytic leukemia, other leukemias), and lymphomas (e.g., B-cell lymphomas, non-Hodgkin's lymphoma). Solid tumors can originate in organs and include cancers of the lungs, brain, breasts, prostate, ovaries, colon, kidneys and liver.

The terms “cancer cell” or “tumor cell” can refer to cells that divide at an abnormal (i.e., increased) rate. Cancer cells include, but are not limited to, carcinomas, such as squamous cell carcinoma, non-small cell carcinoma (e.g., non-small cell lung carcinoma), small cell carcinoma (e.g., small cell lung carcinoma), basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; hematologic cancers, such as myelomas, leukemias (e.g., acute myelogenous leukemia, chronic lymphocytic leukemia, granulocytic leukemia, monocytic leukemia, lymphocytic leukemia), lymphomas (e.g., follicular lymphoma, mantle cell lymphoma, diffuse large B-cell lymphoma, malignant lymphoma, plasmocytoma, reticulum cell sarcoma, or Hodgkin's disease), and tumors of the nervous system including glioma, glioblastoma multiform, meningoma, medulloblastoma, schwannoma and epidymoma.

The term “nanoparticle” refers to any particle having a diameter of less than 1000 nanometers (nm). In general, the nanoparticles should have dimensions small enough to allow their uptake by eukaryotic cells. Typically, the nanoparticles have a longest straight dimension (e.g., diameter) of 200 nm or less. In some embodiments, the nanoparticles have a diameter of 100 nm or less. Smaller nanoparticles, e.g., having diameters of about 60 nm or less are used in some embodiments.

The phrases “parenteral administration” and “administered parenterally” are art-recognized terms and include modes of administration other than enteral and topical administration, such as injections, and include, without limitation, intratumoral, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrastemal injection and infusion.

The phrases “systemic administration,” “administered systemically,” “peripheral administration” and “administered peripherally” as used herein mean the administration of a compound, agent or other material other than directly into a specific tissue, organ, or region of the subject being treated (e.g., tumor site), such that it enters the animal's system and, thus, is subject to metabolism and other like processes, for example, subcutaneous administration.

A “nucleic acid” refers to a polynucleotide and includes polyribonucleotides and polydeoxyribonucleotides.

“Treating”, as used herein, means ameliorating the effects of, or delaying, halting or reversing the progress of a disease or disorder. The word encompasses reducing the severity of a symptom of a disease or disorder and/or the frequency of a symptom of a disease or disorder.

A “subject”, as used therein, can be a human or non-human animal. Non-human animals include, for example, livestock and pets, such as ovine, bovine, porcine, canine, feline and murine mammals, as well as reptiles, birds and fish. Preferably, the subject is human.

The language “effective amount” or “therapeutically effective amount” refers to a sufficient amount of the composition used in the practice of the invention that is effective to provide effective treatment in a subject, depending on the compound being used. That result can be reduction and/or alleviation of the signs, symptoms, or causes of a disease or disorder, or any other desired alteration of a biological system. An appropriate therapeutic amount in any individual case may be determined by one of ordinary skill in the art using routine experimentation.

A “prophylactic” or “preventive” treatment is a treatment administered to a subject who does not exhibit signs of a disease or disorder or exhibits only early signs of the disease or disorder, for the purpose of decreasing the risk of developing pathology associated with the disease or disorder.

A “therapeutic” treatment is a treatment administered to a subject who exhibits signs of pathology of a disease or disorder for the purpose of diminishing or eliminating those signs.

“Pharmaceutically acceptable carrier” refers herein to a composition suitable for delivering an active pharmaceutical ingredient, such as the composition of the present invention, to a subject without excessive toxicity or other complications while maintaining the biological activity of the active pharmaceutical ingredient. Protein-stabilizing excipients, such as mannitol, sucrose, polysorbate-80 and phosphate buffers, are typically found in such carriers, although the carriers should not be construed as being limited only to these compounds.

The terms “homology” and “identity” are used synonymously throughout and refer to sequence similarity between two peptides or between two nucleic acid molecules. Homology can be determined by comparing a position in each sequence, which may be aligned for purposes of comparison. When a position in the compared sequence is occupied by the same base or amino acid, then the molecules are homologous or identical at that position. A degree of homology or identity between sequences is a function of the number of matching or homologous positions shared by the sequences.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

Embodiments described herein relate to immunostimulatory nanoparticles (immuno-NPs) and their use in pharmaceutical compositions for the treatment of cancer. The immuno-NPs can be loaded with a Toll-like Receptor 9 (TLR9) agonist and a nucleic acid inhibitor of V domain Immunoglobulin Suppressor of T cell activation (VISTA, also known as PD-1H, DD1a, or Dies1). It was found that the use of immuno-NPs loaded with agents simultaneously targeting VISTA and activating TLR9 provides a synergistic effect on the activation of innate immune cells into T cell-stimulator cells driving antitumor immune responses to recruit systemic immunity from within the tumor itself. It is believed that the effective delivery to tumor resident myeloid cells (e.g., myeloid-derived suppressor cells (MDSCs), dendritic cells (DCs), and macrophages) of these two synergistic agents of the immuno-NPs reprograms and activates the local myeloid cells into T cell-stimulatory cells capable of priming tumor-reactive cytotoxic CD8+ T cells, thereby eliciting T-cell mediated killing of tumor cells with dimished systemic toxicity.

The immuno-NPs nanoparticles can be made from any biocompatible, biodegradable material that can include or be loaded with the TLR9 agonist and the nucleic acid inhibitor of VISTA, shield the TLR9 agonist and the nucleic acid inhibitor of VISTA from degradation upon administration of the immuno-NPs to the subject, and release the TLR9 agonist and the nucleic acid inhibitor of VISTA upon internalization of the nanoparticles by a myeloid cell. Examples of nanoparticles can include liposomes, lipidic nanoparticles, a hydrogel, micelles, polymer nanoparticles, dendrimers, and/or combinations of these materials.

The immuno-NPs can have a maximum size or diameter of about 20 nm to about 1 μm, preferably about 30 nm to about 100 nm, or more preferably about 30 nm to about 60 nm. In general, the immuno-NPs can have dimensions small enough to allow the immuno-NPs to be directly, locally, or systemically administered to a subject and targeted to cells, tissue, and/or disease sites of the subject. In some embodiments, the immuno-NPs can have a size that facilitates encapsulation of the TLR9 agonist and the nucleic acid inhibitor of VISTA.

The immuno-NPs may be uniform (e.g., being about the same size) or of variable size. Particles may be any shape (e.g., spherical or rod shaped), but are preferably made of regularly shaped material (e.g., spherical). Other geometries can include substantially spherical, circular, triangle, quasi-triangle, square, rectangular, hexagonal, oval, elliptical, rectangular with semi-circles or triangles and the like. Selection of suitable materials and geometries are known in the art.

In some embodiments, the immuno-NPs can be lipid nanoparticles or, liposomes that include a biocompatible lipid shell that defines an outer surface of the nanoparticle and a core, which is loaded with the TLR9 agonist and the nucleic acid inhibitor of VISTA. The biocompatible lipid shell of the immuno-NPs can be configured to shield the TLR9 agonist and the nucleic acid inhibitor of VISTA from degradation upon administration of the immuno-NPs to the subject and release the TLR9 agonist and the nucleic acid inhibitor of VISTA upon internalization of the nanoparticle by a myeloid cell.

In some embodiments, the lipid shell can include one or more ionizable cationic lipids that form liposome-like structures capable of encapsulating a broad variety of anionic nucleic acids (RNA and DNA). Immuno-NPs having lipid shells of ionizable cationic lipids can have several features for the systemic delivery of polynucleic acids, including small sizes, serum stability, low surface zeta potentials at physiological pH, and cationic charge at acidic pH values (e.g., in endosomes). Ionizable cationic lipids can also promote endosome escape and/or reduce toxicity of the immuno-NPs.

In some embodiments, the ionizable cationic lipid can include, for example, D-Lin-MC3-DMA (also known as MC3 or (6Z,9Z,28Z,31Z)-Heptatriaconta-6,9,28,31-tetraen-19-yl 4-(dimethylamino) butanoate), and analogs thereof. Additional ionizable cationic lipids for use in a biocompatible lipid nanoparticle described herein can include, but are limited to, ALC-0315, SM-102, 7-[(2-Hydroxyethyl) [8-(nonyloxy)-8-oxooctyl]amino]heptyl 2-octyldecanoate, DODMA, DODAP, DLin-KC2-DMA, C12-200, BP-Lipid 215, Lipid H (SM⁻¹⁰²) and analogs thereof.

The lipd shell of the immuno-NPs can include additional lipid ingredients derived from naturally-occuring, synthetitic or semi-synthetic (i.e., modified natural) material. In some embodiments, the lipid shell can include naturally-occurring, synthetic or semi-synthetic material that is generally amphipathic (i.e., including a hydrophilic component and a hydrophobic component). Examples of materials that can be used to form the lipid shell of the immune-NP include other lipids, fatty acids, neutral fats, phospholipids, oils, glycolipids, surfactants, aliphatic alcohols, waxes, terpenes and steroids as well as semi-synthetic or modified natural lipids. Semi-synthetic or modified natural lipids can include natural lipids that have been chemically modified in some fashion. The lipid can be neutrally-charged, negatively-charged (i.e., anionic), or positively-charged (i.e., cationic).

Other examples of lipids, any one or combination of which may be used to form the shell of the immuno-NP described herein, can include: phosphocholines, such as 1-alkyl-2-acetoyl-sn-glycero 3-phosphocholines, and 1-alkyl-2-hydroxy-sn-glycero 3-phosphocholines; phosphatidylcholine with both saturated and unsaturated lipids, including dioleoylphosphatidylcholine, dimyristoylphosphatidylcholine, dipentadecanoylphosphatidylcholine, dilauroylphosphatidylcholine, dipalmitoylphosphatidylcholine (DPPC), distearoylphosphatidylcholine (DSPC), and diarachidonylphosphatidylcholine (DAPC); phosphatidylethanolamines, such as dioleoylphosphatidylethanolamine, dipalmitoylphosphatidylethanolamine (DPPE), and distearoylphosphatidylethanolamine (DSPE); phosphatidylserine; phosphatidylglycerols, including distearoylphosphatidylglycerol (DSPG); phosphatidylinositol; sphingolipids, such as sphingomyelin; glycolipids, such as ganglioside GM1 and GM2; glucolipids; sulfatides; glycosphingolipids; phosphatidic acids, such as dipalmitoylphosphatidic acid (DPPA) and distearoylphosphatidic acid (DSPA); palmitic acid; stearic acid; arachidonic acid; oleic acid; lipids bearing polymers, such as chitin, hyaluronic acid, polyvinylpyrrolidone or polyethylene glycol (PEG); lipids bearing sulfonated mono-, di-, oligo- or polysaccharides; cholesterol, cholesterol sulfate, and cholesterol hemisuccinate; tocopherol hemisuccinate; lipids with ether and ester-linked fatty acids; polymerized lipids (a wide variety of which are well known in the art); diacetyl phosphate; dicetyl phosphate; stearylaamine; cardiolipin; phospholipids with short chain fatty acids of about 6 to about 8 carbons in length; synthetic phospholipids with asymmetric acyl chains, such as, for example, one acyl chain of about 6 carbons and another acyl chain of about 12 carbons; ceramides; non-ionic liposomes including niosomes, such as polyoxyalkylene (e.g., polyoxyethylene) fatty acid esters, polyoxyalkylene (e.g., polyoxyethylene) fatty alcohols, polyoxyalkylene (e.g., polyoxyethylene) fatty alcohol ethers, polyoxyalkylene (e.g., polyoxyethylene) sorbitan fatty acid esters (such as, for example, the class of compounds referred to as TWEEN (commercially available from ICI Americas, Inc., Wilmington, DE), glycerol polyethylene glycol oxystearate, glycerol polyethylene glycol ricinoleate, alkyloxylated (e.g., ethoxylated) soybean sterols, alkyloxylated (e.g., ethoxylated) castor oil, polyoxyethylene-polyoxypropylene polymers, and polyoxyalkylene (e.g., polyoxyethylene) fatty acid stearates; sterol aliphatic acid esters including cholesterol sulfate. cholesterol butyrate, cholesterol isobutyrate, cholesterol palmitate, cholesterol stearate, lanosterol acetate, ergosterol palmitate, and phytosterol n-butyrate; sterol esters of sugar acids including cholesterol glucuronide, lanosterol glucuronide, 7-dehydrocholesterol glucuronide, ergosterol glucuronide, cholesterol gluconate, lanosterol gluconate, and ergosterol gluconate; esters of sugar acids and alcohols including lauryl glucuronide, stearoyl glucuronide, myristoyl glucuronide, lauryl gluconate, myristoyl gluconate, and stearoyl gluconate; esters of sugars and aliphatic acids including sucrose laurate, fructose laurate, sucrose palmitate, sucrose stearate, glucuronic acid, gluconic acid and polyuronic acid; saponins including sarsasapogenin, smilagenin, hederagenin, oleanolic acid, and digitoxigenin; glycerol dilaurate, glycerol trilaurate, glycerol dipalmitate, glycerol and glycerol esters including glycerol tripalmitate, glycerol distearate, glycerol tristearate, glycerol dimyristate, glycerol trimyristate; long chain alcohols including n-decyl alcohol, lauryl alcohol, myristyl alcohol, cetyl alcohol, and n-octadecyl alcohol; 6-(5-cholesten-3β-yloxy)-1-thio-β-D-galactopyranoside; digalactosyldiglyceride; 6-(5-cholesten-3ß-yloxy) hexyl-6-amino-6-deoxy-1-thio-β-D-galactopyranoside; 6-(5-cholesten-3β-yloxy) hexyl-6-amino-6-deoxyl-1-thio-α-D-mannopyranoside; 12-(((7′-diethylaminocoumarin-3-yl) carbonyl)methylamino) octadecanoic acid; N-[12-(((7′-diethylaminocoumarin-3-yl) carbonyl)methylamino) octadecanoyl]-2-aminopalmitic acid; cholesteryl (4′-trimethylammonio) butanoate; N-succinyldioleoylphosphatidylethanolamine; 1,2-dioleoyl-sn-glycerol; 1,2-dipalmitoyl-sn-3-succinylglycerol; 1,3-dipalmitoyl-2-succinylglycerol; 1-hexadecyl-2-palmitoylglycerophosphoethanolamine and palmitoylhomocysteine; and/or any combinations thereof.

In certain embodiments, these additional lipid ingredients used to form the membrane or shell of the immune-NPs described herein can include neutral phospholipid molecules belonging to the phosphatidylcholine (PC) class and sterols, such as cholesterol. In some embodiments, a PEGylated phospholipid (a polyethylene glycol (PEG) polymer covalently attached to the head-group of a phospholipid) can be used to form the lipid shell of the immuno-NP described herein. PEGylated phospholipid can be added to the immuno-NP described herein to increase bloodstream circulation lifetime and/or to increase the stability of the nanoparticle compositions.

In certain embodiments, lipid shells of the immuno-NPs can include a lipid mixture of an ionizable cationic lipid, cholesterol, and at least one additional lipid. In one example, the lipid shells of the immuno-NPs can can include about 42 mol % to about 50 mol % of an ionizable cationic lipid, about 38 mol % to about 40 mol % cholesterol, and about 11 mol % to about 14 mol % of a phospholipid. In another example, the lipid shells of the immuno-NPs can include about 42 mol % to about 50 mol % of an ionizable cationic lipid, about 10.5 mol % to about 11 mol % DSPC, about 38 mol % to about 40 mol % cholesterol, 1 mol % to about 5 mol % DMG-PEG, and about 0.5 mol % to about 2.3 mol % DSPE-PEG. In yet another example, the lipid shells of the immuno-NPs can include about 50 mol % MC3, about 10.5 mol % DSPC, about 38 mol % cholesterol, about 1.4 mol % DMG-PEG and about 0.1 mol % DSPE-PEG.

In some embodiments, the nucleic acid inhibitor of VISTA loaded in the immuno-NPs is an antisense nucleic acid to a coding region of VISTA. An “antisense” nucleic acid comprises a nucleotide sequence which is complementary to a “sense” nucleic acid encoding a polypeptide, e.g., complementary to the coding strand of a double-stranded cDNA molecule or complementary to an mRNA sequence. Accordingly, an antisense nucleic acid can hydrogen bond to a sense nucleic acid.

The antisense nucleic acid can be complementary to an entire VISTA coding strand, or to only a portion thereof. In one embodiment, an antisense nucleic acid molecule is antisense to a “coding region” of the coding strand of a nucleotide sequence encoding a VISTA, such as SEQ ID NO: 1 or SEQ ID NO: 2. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding VISTA. The term “noncoding region” refers to 5′ and 3′ sequences which flank the coding region that are not translated into amino acids (also referred to as 5′ and 3′ untranslated regions).

Given the coding strand sequences encoding human VISTA or mouse VISTA, antisense nucleic acids of the invention can be designed according to the rules of Watson and Crick base pairing. The antisense nucleic acid molecule can be complementary to the entire coding region of VISTA mRNA, but more preferably is an oligonucleotide which is antisense to only a portion of the coding or noncoding region of VISTA mRNA. For example, the antisense oligonucleotide can be complementary to the region surrounding the translation start site of VISTA or VISTA mRNA. An antisense oligonucleotide can be, for example, about 5, 10, 15, 20, 25, 30, 35, 40, 45 or 50 nucleotides in length.

In some embodiments, an antisense nucleic acid molecule described herein can be constructed using chemical synthesis and enzymatic ligation reactions using procedures known in the art. For example, an antisense nucleic acid molecule (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridin-e, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3) w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules described herein are typically generated such that they hybridize with or bind to cellular mRNA and/or genomic DNA encoding a VISTA or VISTA polypeptide to thereby inhibit expression of the polypeptide, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix.

The VISTA antisense nucleic acid molecule may be an α-anomeric nucleic acid molecule. An α-anomeric nucleic acid molecule forms specific double-stranded hybrids with complementary RNA in which, contrary to the usual β-units, the strands run parallel to each other. Gaultier, et al. (1987) Nucleic Acids Res. 15:6625-6641. The antisense nucleic acid molecule can also comprise a 2′-O-methylribonucleotide (Inoue, et al. (1987) Nucleic Acids Res. 15:6131-6148) or a chimeric RNA-DNA analogue (Inoue, et al. (1987) FEBS Lett. 215:327-330).

A VISTA antisense nucleic acid may be a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haseloff and Gerlach (1988) Nature 334:585-591)) can be used to catalytically cleave VISTA mRNA transcripts to thereby inhibit translation of VISTA mRNA. A ribozyme having specificity for a VISTA-encoding nucleic acid can be designed based upon the nucleotide sequence of a VISTA cDNA. For example, a derivative of a Tetrahymena L-19 IVS RNA can be constructed in which the nucleotide sequence of the active site is complementary to the nucleotide sequence to be cleaved in a VISTA-encoding mRNA. See, e.g., U.S. Pat. Nos. 4,987,071 and 5,116,742. Alternatively, VISTA mRNA can be used to select a catalytic RNA having a specific ribonuclease activity from a pool of RNA molecules. See, e.g., Bartel and Szostak (1993) Science 261:1411-1418

Alternatively, VISTA gene expression can be inhibited by targeting nucleotide sequences complementary to the regulatory region of the VISTA (e.g., the VISTA promoter and/or enhancers; to form triple helical structures that prevent transcription of the PD-L3 gene in target cells. See generally, Helene (1991) Anticancer Drug Des. 6 (6): 569-84; Helene, et al. (1992) Ann. N.Y. Acad. Sci. 660:27-36; and Maher, L. J. (1992) Bioessays 14 (12): 807-15.

In yet another embodiment, the VISTA antisense molecules can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids. See Hyrup and Nielsen (1996) Bioorg. Med. Chem. 4 (1): 5-23. As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup and Nielsen (1996) supra and Perry-O'Keefe et al. (1996) Proc Natl. Acad. Sci. USA 93:14670-675.

PNAs of VISTA nucleic acids can be used as antisense or antigene agents for sequence-specific modulation of gene expression by, for example, inducing transcription or translation arrest or inhibiting replication.

PNAs of VISTA can be modified (e.g., to enhance their stability or cellular uptake), by attaching lipophilic or other helper groups to PNA, by the formation of PNA-DNA chimeras prior to incorporation in the nanoparticle. For example, PNA-DNA chimeras of VISTA nucleic acid molecules can be generated which may combine the advantageous properties of PNA and DNA. Such chimeras allow DNA recognition enzymes (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup and Nielsen (1996) supra). The synthesis of PNA-DNA chimeras can be performed as described in Hyrup and Nielsen (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxythymidine phosphoramidite, can be used as a bridge between the PNA and the 5′ end of DNA (Mag, M. et al. (1989) Nucleic Acids Res. 17:5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, et al. (1975) Bioorganic Med. Chem. Lett. 5:1119-11124).

In some embodiments, a nucleic acid inhibitor of VISTA is an RNAi construct. RNAi constructs for use in compositions or methods described herein can comprise double stranded RNA that can specifically block expression of a target gene, e.g., the C10orf54 gene encoding VISTA. As used herein, the term “RNAi construct” is a generic term used throughout the specification to include small interfering RNAs (siRNAs), hairpin RNAs, and other RNA species, which can be cleaved in vivo to form siRNAs.

“RNA interference” or “RNAi” is a term initially applied to a phenomenon observed in plants and worms where double-stranded RNA (dsRNA) blocks gene expression in a specific and post-transcriptional manner. As used herein, the term “dsRNA” refers to siRNA molecules or other RNA molecules including a double stranded feature and able to be processed to siRNA in cells, such as hairpin RNA moieties

In some embodiments, the RNAi constructs inhibiting VISTA loaded into of an immune-NP as described herein can decrease the expression of a therapeutic target in the cell of a subject in need thereof using gene silencing. It was shown that successful gene silencing of VISTA can reprogram the immunosuppressive innate immune cells (e.g., DCs and MDSCs) to anti-tumor immunity, and VISTA blockade combined with TLR9 activation delivered using immune-NPs elicit functional synergy.

The RNAi constructs contain a nucleotide sequence that hybridizes under physiologic conditions of the cell to the nucleotide sequence of at least a portion of the mRNA transcript for the gene to be inhibited. The double-stranded RNA need only be sufficiently similar to natural RNA that it has the ability to mediate RNAi. As used herein, the phrase “mediates RNAi” refers to (indicates) the ability to distinguish which RNAs are to be degraded by the RNAi process, e.g., degradation occurs in a sequence-specific manner rather than by a sequence-independent dsRNA response, e.g., a PKR response.

Thus, embodiments described herein tolerate sequence variations that might be expected due to genetic mutation, strain polymorphism or evolutionary divergence. The number of tolerated nucleotide mismatches between the target sequence and the RNAi construct sequence can be no more than 1 in 5 basepairs, or 1 in 10 basepairs, or 1 in 20 basepairs, or 1 in 50 basepairs. Mismatches in the center of the RNAi duplex are most critical and may essentially abolish cleavage of the target RNA. In contrast, nucleotides at the 3′ end of the RNAi strand that is complementary to the target RNA do not significantly contribute to specificity of the target recognition.

Sequence identity may be optimized by sequence comparison and alignment algorithms known in the art and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript.

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of a nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis. A modified ribonucleotide can be introduced by in vitro enzymatic or organic synthesis.

Methods of chemically modifying RNA molecules can be adapted for modifying RNAi constructs (see for example, Nucleic Acids Res, 25:776-780; J Mol Recog 7:89-98; Nucleic Acids Res 23:2661-2668; Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodie-sters, peptide nucleic acids, 5-propynyl-pyrimidine containing oligomers or sugar modifications (e.g., 2′-substituted ribonucleosides, a-configuration).

The double-stranded structure may be formed by a single self-complementary RNA strand or two complementary RNA strands. RNA duplex formation may be initiated either inside or outside the cell. The RNA may be introduced in an amount, which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

In certain embodiments, the RNAi constructs for use as a nucleic acid inhibitor of VISTA are “small interfering RNAs” or “siRNAs”. SiRNAs are a class of double-stranded RNA molecules usually about 20-25 nucleotides in length that bind to a specific mRNA and direct it to mRNA degradation, thus suppressing the transcription (e.g., expression) of the gene. See Hamilton & Baulcombe (1999) Science 286 (5441): 950-2 and Elbashir, et al. (2001) Nature 411 (6836): 494-8. It is also possible to take advantage of ribozyme or RNA interference (siRNA) technology, which prevents a gene from producing a functional protein by destroying the messenger RNA. An siRNA molecule may bind to VISTA mRNA transcribed from a VISTA.

An siRNA molecule which targets VISTA mRNA transcribed from a VISTA DNA may comprise a 10 to 30 nucleic acid portion of the nucleic acid sequence of SEQ ID NO: 1 or 2. In some embodiments, the siRNA molecule that targets VISTA may comprise the nucleic acid sequence of any one of SEQ ID NOs: 3-32. In other embodiments, the siRNA molecule that targets either the ORF or UTR region of VISTA may comprise the nucleic acid sequence of any one of SEQ ID NO: 3-12. In other embodiments, teh siRNA molecule that targets the UTR region only of VISTA may comprise the nucleic acid sequence of any one of SEQ ID NO: 13-22. In other embodiments, the siRNA molecule that targets the ORF region only of VISTA may comprise the nucleic acid sequence of any one of SEQ ID NO: 23-32. In other embodiments, the siRNA molecule that targets VISTA may consist of the nucleic acid sequence of any one of SEQ ID NOs: 3-32. In other embodiments, the siRNA molecule that targets either the ORF or UTR region of VISTA may consist of the nucleic acid sequence of any one of SEQ ID NO: 3-12. In other embodiments, the siRNA molecule that targets the UTR region only of VISTA may consist of the nucleic acid sequence of any one of SEQ ID NO: 13-22. In other embodiments, the siRNA molecule that targets the ORF region only of VISTA may consist of the nucleic acid sequence of any one of SEQ ID NO: 23-32.

TABLE 1
siRNA for human VISTA

GGGCACGATGTGACCTTCTACAAGA (SEQ ID NO: 3)
CAGATGCCAAATGACTTACATCTTA (SEQ ID NO: 4)
GAGATGGATTGTAAGAGCCAGTTTA (SEQ ID NO: 5)
GGGCTTTGAGGAGAGGGTAAACATA (SEQ ID NO: 6)
CCTATCTCCTGACATTCACAGTTTA (SEQ ID NO: 7)
CAGTTTAATAGAGACTTCCTGCCTT (SEQ ID NO: 8)
CAGGGAGAGGCTGAAGGAATGGAAT (SEQ ID NO: 9)
GGATTGTGTTGAGAGGGATTCTGAA (SEQ ID NO: 10)
GAGAGGGATTCTGAATGATCAATAT (SEQ ID NO: 11)
CACAGAGGGCAATAGAGGTTCTGAA (SEQ ID NO: 12)
CAGATGCCAAATGACTTACATCTTA (SEQ ID NO: 13)
GAGATGGATTGTAAGAGCCAGTTTA (SEQ ID NO: 14)
GGTGAGTCCTCTGTGGAATTGTGAT (SEQ ID NO: 15)
GGGCTTTGAGGAGAGGGTAAACATA (SEQ ID NO: 16)
CCTATCTCCTGACATTCACAGTTTA (SEQ ID NO: 17)
CAGTTTAATAGAGACTTCCTGCCTT (SEQ ID NO: 18)
CAGGGAGAGGCTGAAGGAATGGAAT (SEQ ID NO: 19)
GGAATGTGTTGAGAGGGATTCTGAA (SEQ ID NO: 20)
GAGAGGGATTCTGAATGATCAATAT (SEQ ID NO: 21)
CACAGAGGGCAATAGAGGTTCTGAA (SEQ ID NO: 22)
ACAAAGGGCACGATGTGACCTTCTA (SEQ ID NO: 23)
GGGCACGATGTGACCTTCTACAAGA (SEQ ID NO: 24)
GACCACCATGGCAACTTCTCCATCA (SEQ ID NO: 25)
CAGACAGGCAAAGATGCACCATCCA (SEQ ID NO: 26)
GGCAAAGATGCACCATCCAACTGTG (SEQ ID NO: 27)
CCATCCAACTGTGTGGTGTACCCAT (SEQ ID NO: 28)
GGATGGACAGCAACATTCAAGGGAT (SEQ ID NO: 29)
GACAGCAACATTCAAGGGATTGAAA (SEQ ID NO: 30)
CCCTGTCCCTGACTCTCCAAACTTT (SEQ ID NO: 31)
CCTGACTCTCCAAACTTTGAGGTCA (SEQ ID NO: 32)

The siRNA molecules described herein can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end (Proc Natl Acad Sci USA, 98:9742-9747; EMBO J, 20:6877-88). These double-stranded siRNA structures can then be directly introduced to cells, either by passive uptake or a delivery system of choice, such as described below.

In certain embodiments, the siRNA constructs can be generated by processing of longer double-stranded RNAs, for example, in the presence of the enzyme dicer to produce a dicer-substrate siRNA. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

In some embodiments, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Genes Dev, 2002, 16:948-58; Nature, 2002, 418:38-9; RNA, 2002, 8:842-50; and Proc Natl Acad Sci, 2002, 99:6047-52. Such hairpin RNAs are engineered in cells or in an animal to ensure continuous and stable suppression of a desired gene. It is known in the art that siRNAs can be produced by processing a hairpin RNA in the cell.

Immunostimulatory nanoparticles described herein further include a TLR9 agonist loaded in the immune-NP. As used herein, the term “TLR9 agonist” generally refers to an immunostimulatory oligonucleotide compound including a CpG oligonucleotide motif that can enhance or induce an immune stimulation mediated by TLR9. CpG nucleotides are isolated from endogenous sources or synthesized in vivo or in vitro. Examples of sources of endogenous CpG oligonucleotides include, but are not limited to, microorganisms, bacteria, fungi, protozoa, viruses, molds, or parasites. Alternatively, endogenous CpG oligonucleotides are isolated from mammalian benign or malignant neoplastic tumors. Synthetic CpG oligonucleotides can be synthesized in vivo following transfection or transformation of template DNA into a host organism. Alternatively, synthetic CpG oligonucleotides can be synthesized in vitro by polymerase chain reaction (PCR) or other art-recognized methods (Sambrook, J., Fritsch, E. F., and Maniatis, T., Molecular Cloning: A Laboratory Manual. Cold Spring Harbor Laboratory Press, NY, Vol. 1, 2, 3 (1989), herein incorporated by reference).

Immunostimulatory CpG oligonucleotides can be divided into three types A, B and C, which differ in their immunostimulatory activities. Type A stimulatory CpG oligonucleotides are characterized by a phosphodiester central CpG-containing palindromic motif and a phosphorothioate 3′ poly-G string. Following activation of TLR9, these CpG oligonucleotides induce high IFN-α production from plasmacytoid dendritic cells (pDC). Type A CpG oligonucleotides weakly stimulate TLR9-dependent NF-κB signaling.

Type B stimulatory CpG oligonucleotides contain a full phosphorothioate backbone with one or more CpG dinucleotides. Following TLR9 activation, these CpG-oligonucleotides strongly activate B cells. In contrast to Type A CpG-ODNs, Type B CpG-ODNS weakly stimulate IFN-α secretion.

Type C stimulatory CpG oligonucleotides comprise features of Types A and B. Type C CpG oligonucleotides contain a complete phosphorothioate backbone and a CpG containing palindromic motif. Similar to Type A CpG ODNs, Type C CpG ODNs induce strong IFN-α production from pDC. Similar to Type B CpG ODNs, Type C CpG ODNs induce strong B cell stimulation.

Exemplary stimulatory CpG oligonucleotides comprise, but are not limited to, ODN 1585, ODN 1668, ODN 1826, ODN 2006, ODN 2006-G5, ODN 2216, ODN 2336, ODN 2395, ODN M362 (all InvivoGen).

In some embodiments, the CpG dinucleotide is selected from the group consisting of CpG, C*pG, CpG*, and C*pG*, wherein C is 2′-deoxycytidine, C* is an analog thereof, G is 2′-deoxyguanosine, and G* is an analog thereof, and p is an internucleoside linkage selected from the group consisting of phosphodiester, phosphorothioate, and phosphorodithioate. In some embodiments C* is selected from the group consisting of 2′-deoxythymidine, arabinocytidine, 2′-deoxythymidine, 2′-deoxy-2′-substituted arabinocytidine, 2′-O-substituted arabinocytidine, 2′-deoxy-5-hydroxycytidine, 2′-deoxy-N4-alkyl-cytidine, 2′-deoxy-4-thiouridine. In some embodiments, G* is 2′ deoxy-7-deazaguanosine, 2′-deoxy-6-thioguanosine, arabinoguanosine, 2′-deoxy-2′substituted-arabinoguanosine, 2′-O-substituted-arabinoguanosine, 2′-deoxyinosine.

The immuno-NPs can be loaded with different ratios of the nucleic acid inhibitor of VISTA (e.g., VISTA siRNA) and TLR9 agonist (e.g., CpG). Depending on the application, the ratio of siRNA and CpG loaded within the immuno-NPs can vary to achieve a desired level of VISTA siliencing and simultaneous activation of tumor resident myeloid cells in a subject. In some embodiments, immuno-NPs described herein can include siRNA targeting VISTA expression and CpG dincucleotides loaded into LNPs at a siRNA/CpG (w/w) ratio of about 10:1 to about 1:10, preferably about 5:1 to about 1:1, for example, about 5:1, about 3:1, and about 1:1. In an exemplary embodiment, an immuno-NP can include at least about 10 nM, about 15 nM, about 20 nM, about 25 nM, about 30 nM, about 35 nM, about 40 nM. about 45 nM, about 50 nM or more VISTA siRNA concentration.

Optionally, the immuno-NPs can include a plurality of targeting moieties linked to the shell and extending from the outer surface that are configured to direct the immuno-NPs to tumor-resident myeloid cells in a tumor microenvironment upon administration of the immuno-NPs to a subject with cancer. The targeting moieties can include any molecule, or complex of molecules, which is/are capable of interacting with a cell surface or extracellular molecule or biomarker of a myeloid cell. The cell surface molecule can include, for example, a cellular protease, a kinase, a protein, a cell surface receptor, a lipid, and/or fatty acid.

In certain embodiments, the targeting moiety specifically binds a cell surface molecule of a target myeloid cell. As used herein, a targeting moiety “specifically binds” to a target molecule if it binds to or associates with the target molecule with an affinity or Ka (that is, an equilibrium association constant of a particular binding interaction with units of 1/M) of, for example, greater than or equal to about 10⁵ M⁻¹. In certain embodiments, the targeting moiety binds to the target molecule with a Ka greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding refers to binding with a Ka of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater. Alternatively, affinity may be defined as an equilibrium dissociation constant (KD) of a particular binding interaction with units of M (e.g., 10⁻⁵ M to 10⁻¹³ M, or less). In certain aspects, specific binding means binding to the target molecule with a K_{D} of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less. The binding affinity of the targeting moiety for the target molecule can be readily determined using conventional techniques, e.g., by competitive ELISA (enzyme-linked immunosorbent assay), equilibrium dialysis, by using surface plasmon resonance (SPR) technology (e.g., the BIAcore 2000 instrument, using general procedures outlined by the manufacturer); by radioimmunoassay; or the like.

In some embodiments, the targeting moiety can include, but is not limited to, synthetic compounds, natural compounds or products, macromolecular entities, bioengineered molecules (e.g., polypeptides, lipids, polynucleotides, antibodies, antibody fragments), and small entities (e.g., small molecules, neurotransmitters, substrates, ligands, hormones and elemental compounds).

In one example, the targeting moiety can comprise an antibody, such as a monoclonal antibody, a polyclonal antibody, or a humanized antibody, including without limitation: Fv fragments, single chain Fv (scFv) fragments, Fab′ fragments, F(ab′) 2 fragments, single domain antibodies, camelized antibodies and antibody fragments, humanized antibodies and antibody fragments, and multivalent versions of the foregoing; multivalent targeting moieties including without limitation: monospecific or bispecific antibodies, such as disulfide Fv fragments, scFv tandems ((scFv) 2 fragments), diabodies, tribodies or tetrabodies, which typically are covalently linked or otherwise stabilized (i.e., leucine zipper or helix stabilized) scFv fragments; and receptor molecules, which naturally interact with a desired target molecule.

The targeting moiety need not originate from a biological source. The targeting moiety may, for example, be screened from a combinatorial library of synthetic peptides. One such method is described in U.S. Pat. No. 5,948,635, incorporated herein by reference, which describes the production of phagemid libraries having random amino acid insertions in the pIII gene of M13. This phage may be clonally amplified by affinity selection.

In certain embodiments, the targeting moiety may comprise a ligand to a receptor molecule, including, for example, ligands that specifically binding to a receptor of a target myeloid cell. Such ligand molecules include ligands that have been modified to increase their specificity of interaction with a target receptor, ligands that have been modified to interact with a desired receptor not naturally recognized by the ligand, and fragments of such ligands.

As discussed in the example, we found that immunosuppressive VISTA+G-MDSC, M-MDSC, M2 macrophages, and mDCs highly overexpress folate receptor β (FRβ). Intratumoral administration of FR-targeted immuno-NPs resulted in predominant cellular uptake by VISTA⁺ myeloid cells, which was significantly higher than untargeted immuno-NPs. The targeting moiety to FRβ overexpressed on VISTA⁺ myeloid cells can include folate as well as antibodies or antigen binding fragments to FRβ.

The targeting moieties can be conjugated to the immuno-NP surface or shell by reacting the targeting moiety with heterobifunctional PEG that is conjugated to one or more lipids in the shell. The PEG molecules can have a variety of lengths and molecular weights, including, for example, PEG 200, PEG 1000, PEG 1500, PEG 4600, PEG 10,000, or combinations thereof.

In some embodiments, the immuno-NPs can be formed by radidly mixing a lipid composition, e.g., about 42 mol % to about 50 mol % of a ionizable cationic lipid, about 10.5 mol % to about 11 mol % DSPC, about 38 mol % to about 40 mol % cholesterol, 1 mol % to about 5 mol % DMG-PEG, and about 0.5 mol % to about 2.3 mol % DSPE-PEG-folate, dissolved in ethanol with siRNA/CpG in acetate buffer at pH 4 (1:3 v/v ethanol to aqueous buffer; N/P ratio of about 6). The suspension can be sonicated for about 1 to about 4 minutes yielding the immuno-NPs with desired sizes of about 30 nm to about 65 nm. The immuno-NP suspension can then be dialyzed against PBS (pH 7.4) to remove residual ethanol and elevate the pH above the pKa of the cationic lipid to generate immuno-NPs with a neutral charge.

In some embodiments, the immuno-NPs can be used in a method of treating cancer in a subject in need thereof. The method can include administering to the subject a therapeutically effective amount of a composition that includes the immuno-NPs described herein. While not intending to be bound by theory, it appears that simultaneously targeting of VISTA and TLR9 ilicits a functional synergy resulting in activation of innate immune cells into T cell-stimulatory cells driving antitumor immune responses to recruit systemic immunity from within the tumor itself. The combination of a TLR9 agonist and a nucleic acid inhibitor of VISTA loaded into the immune-NPs also allows for a lower systemic dose when compared to administering each agent alone, thereby mitigating the impact of adverse events, such as those typical of systemic cancer therapies.

Cancers treated by a method described herein can include the following: leukemias, such as but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome; chronic leukemias, such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, glioblastoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to ductal carcinoma, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytoma and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, fallopian tube cancer, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, prostatic intraepithelial neoplasia, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypemephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

Many of the major cancers occurring in human subjects frequently metastasize, including breast, bladder, colon, kidney, melanoma, and prostate. Therefore, in some embodiments, the compositions described herein are used to treat a metastatic cancer selected from the group consisting of but not limited to melanoma, breast cancer, bladder cancer, kidney cancer, colon cancer, lung cancer, prostate cancer and ovarian cancer. For example, in some embodiments, compositions described herein are used to treat metastatic cancer which has spread to one or more sites beyond the initial point where cancer has occurred. In particular embodiments, the cancer treated in accordance with a method described herein can include a cancer characterized by tumors with low immunogenicity, such as ovarian cancer, colon cancer, or melanoma. In particular embodiments, the compositions are used to treat metastatic melanoma.

In some embodiments, a method of treating cancer includes administering the immuno-NP compositions described herein in combination with an immune checkpoint therapy. Immune checkpoint therapy for cancer encompasses strategies that target immunity regulatory pathways in order to enhance immunity activity against tumor cells. In some embodiments, the immune checkpoint therapy can include the administration to a subject of one or more immune checkpoint modulating agents. The immune checkpoint modulating agents can target the same immune checkpoint or can target two or more immune checkpoints.

An immune checkpoint modulating agent for use in a method described herein can include an agent that either inhibits negative regulators of the immune system response against cancer cells, such as programmed cell death protein 1 (PD-1), or agents that act as agonists for positive regulators, such as OX40 (CD134). In some embodiments, the immune checkpoint modulating agents can include an immune checkpoint-targeting antibody such as anti-PD-1 or agonistic OX40-specific monoclonal antibodies.

The programmed death 1 (PD-1) immune checkpoints are negative regulators of T-cell immune function and inhibition of PD-1, results in increased activation of the immune system. In some embodiments, an immune checkpoint modulating agent administered to a subject can include a PD-1 inhibitor. In certain embodiments, the PD-1 inhibitor is an anti-PD-1 antibody. Anti-PD-1 antibodies for use in a method described herein can include monoclonal antibodies capable of inhibiting the engagement/interaction of PD-1 with PD-L1 ligand. Thus, in some embodiments, a PD-1 inhibitory agent can include an antibody that targets PD-L1.

Examples of PD-1 inhibitory monoclonal antibodies for use in a combination therapy described herein include, but are not limited to, Pembrolizumab, Nivolumab, and Cemiplimab, and MEDI0608. Examples of PD-L1 inhibitory monoclonal antibodies include, but are not limited to, Atezolizumab, Avelumab, Vonlerolizumab, and Durvalumab.

In some embodiments, the additional immune checkpoint modulating agent is another agent capable of inhibiting or blocking engagement/interaction with VISTA. In addition to the nucleic acid inhibitor of VISTA, agents targeting VISTA can include human monoclonal antibody JNJ-61610588 and CA-170, an oral inhibitor of both PD-L1/PD-L2 and VISTA.

Additional examples of immune checkpoint modulating agents that target negative regulators of the immune system response against cancer cells can include agents capable of inhibiting or blocking engagement/interaction with Cytotoxic T-lymphocyte-associated antigen 4 (CTLA-4), lymphocyte activation gene 3 (LAG-3, CD223), T cell immunoglobulin-3 (TIM-3), T cell immunoglobulin and ITIM domain (TIGIT), and B7 homolog 3 (B7/H3).

For example, an immune checkpoint modulating agent targeting CTLA-4 can include the anti-CTLA-4 antibodies Tremelimunab, BMS-986249, and Ipilimumab, which is approved for the treatment of advanced or unresectable melanoma. Agents targeting LAG-3 can include the IMP321 fusion protein and monoclonal antibodies targeting LAG-3, such as Relatlimab or LAG525. An agent targeting TIM-3 can include the anti-TIM-3 monoclonal antibody MBG453. An agent targeting TIGIT can include the anti-TIGIT monoclonal antibody OMP-31M32. Agents targeting B7-H3, also known as CD276, can include Enoblituzumab (MGA271) which is an engineered Fc humanized IgGI monoclonal antibody against B7-H3, the humanized DART protein MGD009, and 8H9 which is an antibody against B7-H3 labeled with radioactive iodine (I-131) which, after internalization, promotes cancer cell death.

In some embodiments, the immune checkpoint modulating agents can include a positive regulator of the immune system response against cancer cells. In some embodiments, immune checkpoint modulating agents that act as positive regulators of the immune system response against cancer cells can include OX40 agonistic agents. In one embodiment, an OX40 agonistic agent can include a monoclonal antibody capable of promoting the engagement/interaction of OX40 with OX40L ligand to promote the NF-κB signaling pathway and T cell clonal expansion and activation. OX40 agnostic agents for use in a method described herein can include, but are not limited to, MEDI6368 fusion protein, MEDI0562, MEDI6469, BMS986178, Pf-04518600 (PF-8600), GSK3174998 and MOXR0916.

Additional immune checkpoint modulating agents for use in a method described herein can include agonistic agents targeting positive regulators of the immune system response against cancer cells such as, but not limited to, Inducible co-stimulator (ICOS), Glucocorticoid-induced TNF receptor family-related protein (GITR), 4-1BB, CD27/CD70 pathway, and CD40. GITR agonists can include TRX-518, an aglycosylated human mAb, BMS-986156, AMG 228, MEDI1873, MK-4166, INCAGN01876, and GWN323. ICOS agonists can include JTX-2011, GSK3359609, and MEDI-570. 4-1BB (CD137) agonists can include Utomilumab (PF-05082566) and Urelumab. Agonists of the CD27/CD70 pathway can include ARGX-110, BMS-936561 (MDX-1203), and Varlilumab. CD40 agonists can include CP-870893, APX005M, ADC-1013, lucatumumab, Chi Lob 7/4, dacetuzumab, SEA-CD40, and RO7009789 monoclonal antibodies.

Additional exemplary immune checkpoint modulating agents can include, but are not limited to, agents capable of inhibiting or blocking engagement/interaction with adenosine A2a receptor (A2aR), CD73, B and T cell lymphocyte attenuator (BTLA, CD272), or non-T cell-associated inhibitory molecules such as transforming growth factor β (TGF-β), Killer immunoglobulin-like receptors (KIRs, CD158), Phosphoinositide 3-kinase gamma (PI3Kγ), and CD47 (integrin-associated protein).

Further immune checkpoint modulating agents can include molecules targeting tumor microenvironment components like Indoleamine 2,3-dioxygenase (IDO), Toll-like receptors (TLRs), IL2R, as well as arginase inhibitors such as CB-1158 or oncolytic peptides, such as LTX-315. For example, agents targeting (IDO) can include BMS-986205, and Indoximod, and the oral agent epacadostat. Agents targeting TLRs for use as an immune checkpoint modulating agent in a method described herein can include MEDI9197, PG545 (pixatimod, pINN), and Polyinosinic-polycytidylic acid polylysine carboxymethylcellulose (poly-ICLC). For example, an IL-2R inhibitory agent can include NKTR-214 (bempeg) and an IL-10 inhibitory agent can include AM0010 (pegilodecakin).

Methods described herein can further include the step of administering a therapeutically effective amount of an additional cancer therapy to the subject. A “cancer therapy”, as used herein, can include any combination of agents or treatment regimen that is capable of negatively affecting cancer in an animal, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of an animal with cancer. Cancer therapeutics can include one or more therapies such as, but not limited to, chemotherapies, radiation therapies, hormonal therapies, and/or biological therapies/immunotherapies. A reduction, for example, in cancer volume, growth, migration, and/or dispersal in a subject may be indicative of the efficacy of a given therapy.

In some embodiments, the method can further include the step of administering a therapeutically effective amount of an additional anticancer therapeutic agent to the subject. The anticancer therapeutic agents can be in the form of biologically active ligands, small molecules, peptides, polypeptides, proteins, DNA fragments, DNA plasmids, interfering RNA molecules, such as siRNAs, oligonucleotides, and DNA encoding for shRNA. In some embodiments, cytotoxic compounds are included in an anticancer agent described herein. Cytotoxic compounds include small-molecule drugs such as doxorubicin, mitoxantrone, methotrexate, and pyrimidine and purine analogs, referred to herein as antitumor agents.

The anticancer therapeutic agent can include an anticancer or an antiproliferative agent that exerts an antineoplastic, chemotherapeutic, antiviral, antimitotic, antitumorgenic, and/or immunotherapeutic effects, e.g., prevent the development, maturation, or spread of neoplastic cells, directly on the tumor cell, e.g., by cytostatic or cytocidal effects, and not indirectly through mechanisms such as biological response modification. There are large numbers of anti-proliferative agent agents available in commercial use, in clinical evaluation and in pre-clinical development. For convenience of discussion, anti-proliferative agents are classified into the following classes, subtypes and species: ACE inhibitors, alkylating agents, angiogenesis inhibitors, angiostatin, anthracyclines/DNA intercalators, anti-cancer antibiotics or antibiotic-type agents, antimetabolites, antimetastatic compounds, asparaginases, bisphosphonates, cGMP phosphodiesterase inhibitors, calcium carbonate, cyclooxygenase-2 inhibitors, DHA derivatives, DNA topoisomerase, endostatin, epipodophylotoxins, genistein, hormonal anticancer agents, hydrophilic bile acids (URSO), immunomodulators or immunological agents, integrin antagonists, interferon antagonists or agents, MMP inhibitors, miscellaneous antineoplastic agents, monoclonal antibodies, nitrosoureas, NSAIDs, ornithine decarboxylase inhibitors, pBATTs, radio/chemo sensitizers/protectors, retinoids, selective inhibitors of proliferation and migration of endothelial cells, selenium, stromelysin inhibitors, taxanes, vaccines, and vinca alkaloids.

The major categories that some anti-proliferative agents fall into include antimetabolite agents, alkylating agents, antibiotic-type agents, hormonal anticancer agents, immunological agents, interferon-type agents, and a category of miscellaneous antineoplastic agents. Some anti-proliferative agents operate through multiple or unknown mechanisms and can thus be classified into more than one category.

Examples of anticancer therapeutic agents that can be administered in combination with a plant virus or virus-like particle described herein include Taxol, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon-α-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin: puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; temozolomide, teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anticancer therapeutic agents include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene: parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; silicon phthalocyanine (PC4) sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosamOinoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

In some embodiments, a method of treating cancer described herein can further include the step of ablating the cancer. Ablating the cancer can be accomplished using a method selected from the group consisting of cryoablation, thermal ablation, radiotherapy, chemotherapy, radiofrequency ablation, electroporation, alcohol ablation, high intensity focused ultrasound, photodynamic therapy, administration of monoclonal antibodies, immunotherapy, and administration of immunotoxins.

In some embodiments, the step ablating the cancer includes immunotherapy of the cancer. Cancer immunotherapy is based on therapeutic interventions that aim to utilize the immune system to combat malignant diseases. It can be divided into unspecific approaches and specific approaches. Unspecific cancer immunotherapy aims at activating parts of the immune system generally, such as treatment with specific cytokines known to be effective in cancer immunotherapy (e.g., IL-2, interferon's, cytokine inducers).

In some embodiments, the step of ablating the cancer includes administering a therapeutically effective amount of radiotherapy (RT) to the subject. In some embodiments, RT is administered prior to administration of the immuno-NP compositions. Radiotherapy uses high-energy rays to treat disease, usually x-rays and similar rays (such as electrons). Radiotherapy administered to a subject can include both external and internal. External radiotherapy (or external beam radiation) aims high-energy x-rays at the tumor site including in some cases the peri-tumor margin. External radiotherapy typically includes the use of a linear accelerator (e.g., a Varian 2100C linear accelerator). External radiation therapy can include three-dimensional conformal radiation therapy (3D-CRT), image guided radiation therapy (IGRT), intensity modulated radiation therapy (IMRT), helical-tomotherapy, photon beam radiation therapy, proton beam radiation therapy, stereotactic radiosurgery and/or sterotactic body radiation therapy (SBRT).

Internal radiotherapy (brachytherapy) involves having radioactive material placed inside the body and allows a higher dose of radiation in a smaller area than might be possible with external radiation treatment. It uses a radiation source that's usually sealed in an implant. Exemplary implants include pellets, seeds, ribbons, wires, needles, capsules, balloons, or tubes. Implants are placed in your body, very close to or inside the tumor. Internal radiotherapy can include intracavitary or interstitial radiation. During intracavitary radiation, the radioactive source is placed in a body cavity (space), such as the uterus. With interstitial radiation, the implants are placed in or near the tumor, but not in a body cavity.

It has been shown that moderate magnetic nanoparticle hyperthermia (mNPH) treatment administered to a tumor can generate immune-based systemic resistance to tumor rechallenge. Therefore, in some embodiments, a therapeutically effective amount of a moderate magnetic nanoparticle hyperthermia (mNPH) treatment can be administered to the subject in combination with an Immuno-NP composition described herein. In some embodiments, a subject is further administered an immune checkpoint modulating agent and/or radiotherapy, wherein the mNPH is activated with an alternating magnetic field (AMF) to produce moderate heat. A mNPH treatment can include the use of a magnetic iron oxide nanoparticle (IONP). Once administered to the subject intratumorally, the mNPH can, in some embodiments, be activated with an alternating magnetic field (AMF) to produce moderate heat (e.g., 43°/60 min) at the tumor site. In some embodiments the RT is hypofractionated RT (HFRT) that delivers larger but fewer doses/fractions than typical RT therapies.

When used in vivo, immuno-NPs alone or in combination with additional therapeutic agents, can be administered as a pharmaceutical composition, comprising a mixture, and a pharmaceutically acceptable carrier. The immuno-NPs may be present in a pharmaceutical composition in an amount from 0.001 to 99.9 wt %, more preferably from about 0.01 to 99 wt %, and even more preferably from 0.1 to 95 wt %.

The immuno-NPs, or pharmaceutical compositions comprising these nanoparticles and/or additional therapeutic agents, may be administered by any method designed to provide the desired effect. Administration may occur enterally or parenterally; for example, intratumorally, orally, rectally, intracisternally, intravaginally, intraperitoneally or locally. Parenteral administration methods include intravascular administration (e.g., intravenous bolus injection, intravenous infusion, intra-arterial bolus injection, intra-arterial infusion and catheter instillation into the vasculature), peri- and intra-target tissue injection, subcutaneous injection or deposition including subcutaneous infusion (such as by osmotic pumps), intramuscular injection, intraperitoneal injection, intracranial and intrathecal administration for CNS tumors, and direct application to the target area, for example by a catheter or other placement device.

In some embodiments, the administration of the composition can be proximal to, or directly adjacent, a tumor site in the subject (e.g., peri-tumoral), directly to the tumor site (e.g., via intratumoral injection), or directly into the tumor draining lymph node(s) to provide a high local concentration of the composition in the tumor microenvironment (TME).

When formulated as separate compositions, “combination therapy” is intended to embrace administration of these therapeutic agents in a sequential manner, that is, wherein each therapeutic agent is administered at a different time, as well as administration of these therapeutic agents, in a substantially simultaneous manner. For example, administration of the immuno-NPs can be carried out in a substantially simultaneous manner as the one or more immune checkpoint modulating agent administration or additional cancer therapeutic. Sequential or substantially simultaneous administration of each therapeutic agent can be effected by any appropriate route including, but not limited to, intratumoral routes, intraperitoneal routes, oral routes, intravenous routes, intramuscular routes, and direct absorption through mucous membrane tissues. A preferred method for administering the immuno-NPs compositions and additional cancer therapeutic agents to a subject having cancer is by peri-tumoral or intratumoral injection. However, the therapeutic agents can be administered by the same route or by different routes. For example, immuno-NPs of the combination selected may be administered by peritumoral or intratumoral injection while the immune checkpoint modulating agent(s) of the combination may be administered orally or intravenously. Alternatively, for example, all therapeutic agents may be administered by peritumoral or intratumoral injection. The sequence in which the therapeutic agents are administered is not narrowly critical.

The immuno-NP compositions can also include, depending on the formulation desired, pharmaceutically acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

Suitable pharmaceutically acceptable carriers may contain inert ingredients which do not unduly inhibit the biological activity of the compounds. The pharmaceutically acceptable carriers should be biocompatible, e.g., non-toxic, non-inflammatory, nonimmunogenic and devoid of other undesired reactions upon the administration to a subject. Standard pharmaceutical formulation techniques can be employed, such as those described in Remington's Pharmaceutical Sciences, ibid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al., “Controlled Release of Biological Active Agents”, John Wiley and Sons, 1986).

Suitable doses can vary widely depending on the therapeutic agent being used. A typical pharmaceutical composition for intravenous administration would be about 0.1 mg to about 10 g per subject per day. However, in other embodiments, doses from about 1 mg to about 1 g, or from about 10 mg to about 1 g can be used. Single or multiple administrations of the compositions may be administered depending on the dosage and frequency as required and tolerated by the subject. In any event, the administration regime should provide a sufficient quantity of the composition of this invention to effectively treat the subject.

The formulations may be conveniently presented in unit dosage form and may be prepared by any of the methods well known in the art of pharmacy. Such methods can include the step of bringing the Immuno-NPs into association with a pharmaceutically acceptable carrier that constitutes one or more accessory ingredients. In general, the formulations are prepared by uniformly and intimately bringing the active agent into association with a liquid carrier, a finely divided solid carrier, or both, and then, if necessary, shaping the product into the desired formulations.

One skilled in the art can readily determine an effective amount of immuno-NP composition and/or additional cancer therapeutic to be administered to a given subject, by taking into account factors such as the size and weight of the subject; the extent of disease penetration; the age, health and sex of the subject; the route of administration; and whether the administration is local or systemic. Those skilled in the art may derive appropriate dosages and schedules of administration to suit the specific circumstances and needs of the subject. For example, suitable doses of the immuno-NPs to be administered can be estimated from the volume of cancer cells to be killed or volume of tumor to which the compositions are being administered.

Useful dosages of the active agents can be determined by comparing their in vitro activity and the in vivo activity in animal models. Methods for extrapolation of effective dosages in mice, and other animals, to humans are known in the art. An amount adequate to accomplish therapeutic or prophylactic treatment is defined as a therapeutically- or prophylactically-effective dose. In both prophylactic and therapeutic regimes, agents are usually administered in several dosages until an effect has been achieved. Effective doses of the immuno-NP compositions described herein can vary depending upon many different factors, including means of administration, target site, physiological state of the patient, whether the patient is human or an animal, characteristics of the subject, such as general health, age, sex, body weight and tolerance to drugs as well as the degree, severity and type of cancer, other medications administered, and whether treatment is prophylactic or therapeutic. The skilled artisan will be able to determine appropriate dosages depending on these and other factors using standard clinical techniques.

Therapeutically effective amounts of immuno-NP compositions and/or an additional cancer therapy can include the amount(s) effective to reprogram and activate local tumor-resident myeloid cells into T cell-stimulatory cells in the subject and promote cytotoxic CD8+ T-cell mediated killing of tumor cells. In some embodiments, a therapeutically effective amount of immuno-NPs and/or additional cancer therapies is the amount required to reduce tumor burden in a subject being treated. In other embodiments, a therapeutically effective amount of an immuno-NP composition described herein is an amount effective to produce a synergistic therapeutic effect at a tumor site of the subject.

The methods described herein contemplate single as well as multiple administrations, given either simultaneously or over an extended period of time. A pharmaceutically acceptable composition including the immuno-NPs, and/or additional cancer therapeutic can be administered at regular intervals, depending on the nature and extent of the cancer's effects, and on an ongoing basis. Administration at a “regular interval,” as used herein, indicates that the therapeutically effective amount is administered periodically (as distinguished from a one-time dose). In one embodiment, the pharmaceutically acceptable composition including the immuno-NPs and/or an additional cancer therapeutic is administered periodically, e.g., at a regular interval (e.g., bimonthly, monthly, biweekly, weekly, twice weekly, daily, twice a day or three times or more often a day). In another embodiment, the pharmaceutically acceptable composition is administered to the subject weekly.

The administration interval for a single individual can be fixed, or can be varied over time, depending on the needs of the individual. For example, in times of physical illness or stress, or if disease symptoms worsen, the interval between doses can be decreased.

For example, the administration of immuno-NPs and/or the additional cancer therapeutic agent can take place at least once on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least once on week 1, 2, 3, 4, 5, 6, 7, 8, 9, 10, 11, 12, 13, 14, 15, 16, 17, 18, 19 or 20, or any combination thereof, using single or divided doses of every 60, 48, 36, 24, 12, 8, 6, 4, or 2 hours, or any combination thereof. Administration can take place at any time of day, for example, in the morning, the afternoon or evening. For instance, the administration can take place in the morning, e.g., between 6:00 a.m. and 12:00 noon; in the afternoon, e.g., after noon and before 6:00 p.m.; or in the evening, e.g., between 6:01 p.m. and midnight.

The following Example has been included to more clearly describe particular embodiments of the invention. However, there are a wide variety of other embodiments within the scope of the present invention, which should not be limited to the particular examples provided herein.

### Example

We designed a highly potent immunostimulatory nanoparticle (immuno-NP) that triggers a robust stimulation of the innate arm of the immune system within a tumor. Our approach of removing the brakes of innate immunity in a tumor by targeting a novel checkpoint protein (called VISTA) specific for innate immune cells can allow expansion of immunotherapies to aggressive and lethal forms of cancer where success is currently limited. The design, fabrication, and application of the nanoparticle, termed immuno-NP or dual-NP is based on a lipid nanoparticle co-loaded with siRNA (for gene silencing of VISTA expression) and the TLR9 agonist CpG (adjuvant). The design of the nanoparticle utilizes optimized pH-dependent ionizable lipids specifically for the dual cargo of VISTA-targeting siRNA and CpG. Simultaneous targeting of VISTA and activation of TLR9 elicit a remarkable functional synergy resulting in activation of innate immune cells into T cell-stimulatory cells driving antitumor immune responses to recruit systemic immunity from within the tumor itself.

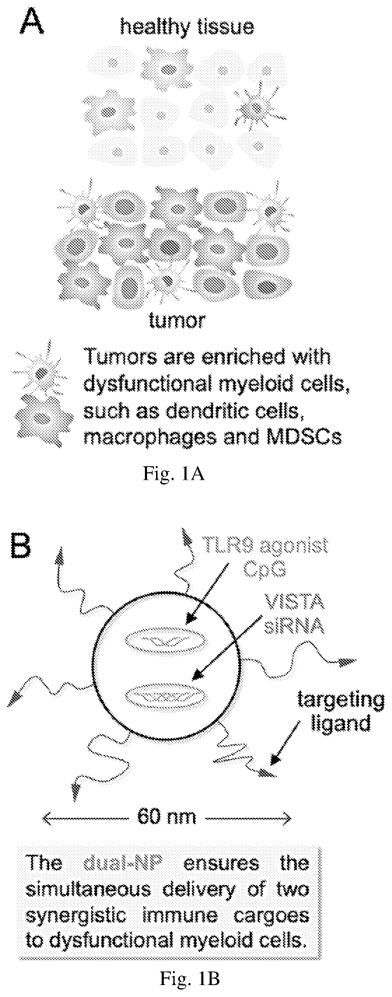

FIG. 1 illustrates a bioengineered immuno-NP or dual-NP used to deliver two synergistic cargoes that reprogram and activate the tumor-resident myeloid cells leading to priming of tumor-reactive cytotoxic CD8+ T cells. We show immuno-NP incorporating effective delivery to tumor-resident myeloid cells, simultaneous and proficient delivery of two synergistic immune cargoes that reprogram and activate tumor-resident myeloid cells into T-cell stimulatory cells, and therapeutic benefits at a low and safe dose.

We show that immuno-NPs resulted in predominant cellular uptake by VISTA+ myeloid cells. Further, upon intratumoral administration, the size of the immuno-NP dictates the spread of the particles throughout the tumor volume and their uptake by the majority of the tumor-resident myeloid cells. We have developed a convenient, consistent and highly controllable method to generate immuno-NPs of different sizes (i.e., 30, 40 or 60 nm) with high degree of uniformity.

FIG. 1B shows the immuno-NP is co-loaded with a VISTA short interfering RNA (siRNA) and the TLR9 agonist CpG. The design utilizes optimized ionizable cationic lipids specifically for the dual cargo of VISTA siRNA and CpG. The immuno-NP provides protection of its two cargoes from degradation, diminished immunogenicity, low systemic toxicity, and effective delivery to the majority of myeloid cells in a tumor. Due to its design, the immuno-NP facilitates highly stable and efficient entrapment of the two oligonucleotides, targeting and preferential uptake by VISTA⁺ myeloid cells (i.e., the desirable target) and proficient presentation of each immune cargo to the appropriate intracellular location (FIG. 1C). Upon internalization of the immuno-NP, the NP collapses leading to direct presentation of CpG to the TLR9-expressing endosomal membrane of myeloid cells. Then, the lower pH and interaction with endosomal lipids leads to cytosolic delivery of the VISTA siRNA payload. We show that the immuno-NP ensures simultaneous delivery of its two synergistic cargoes to dysfunctional myeloid cells, which results in substantial decrease of MDSCs and M2 TAMs and generates a remarkable antitumor immune response resulting in rapid tumor clearance and curative responses with protective immunological memory against tumor recurrence.

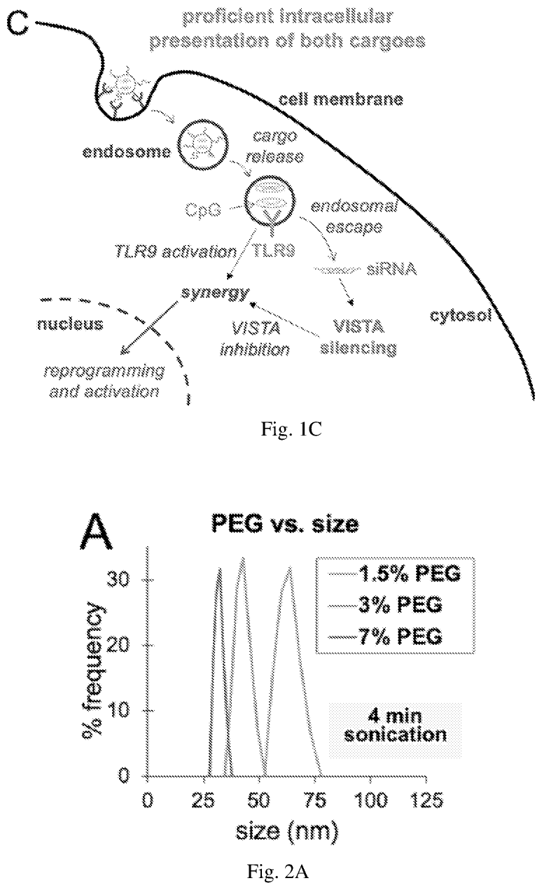

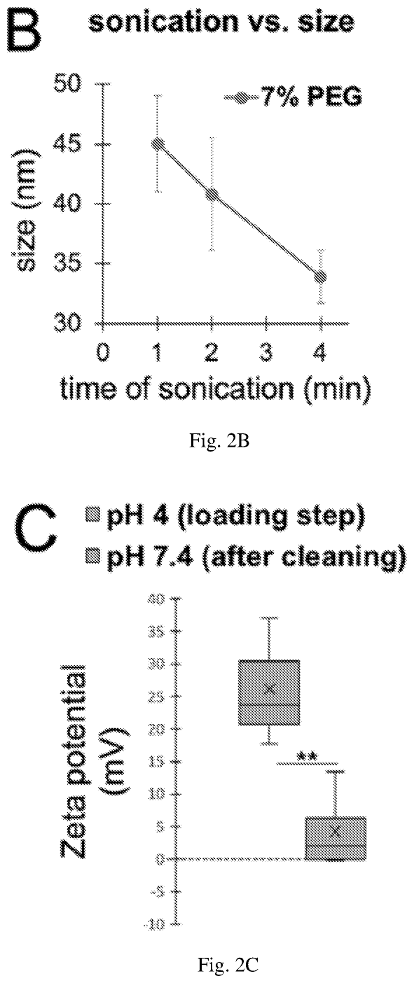

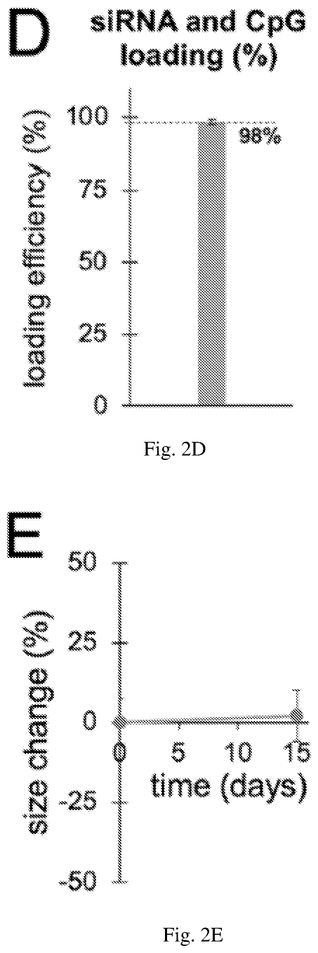

Under mild sonication for short periods of time, the immuno-NPs were formed by rapidly mixing the lipids with siRNA/CpG at pH 4. To accommodate NPs with different sizes, we performed theoretical and experimental analyses to identify the influence of lipid composition and sonication time on the NP size and stability. The lipid composition ranged from 42-50 mol % cationic lipid, 10.5-11 mol % DSPC, 38-40 mol % cholesterol, and 1-5 mol % DMG-PEG. We have used a series of cationic lipids such as MC3 and derivatives of the ionizable Lipid H. Dynamic light scattering (DLS) measurements indicated that the immuno-NP variants exhibited different sizes with very narrow size distributions (FIG. 2A). The effect of sonication time is shown in FIG. 2B. Zeta potential measurements of the formulation indicated a nearly neutral surface charge (FIG. 2C), while high amounts of CpG and VISTA siRNA were conveniently and stably loaded in the immuno-NP (FIG. 2D). It should be noted that the fabrications conditions were mild and therefore we measured no loss of activity or damage of the NP. Stability tests indicated immuno-NPs remained unaltered over the period of few weeks (FIG. 2E).

Immuno-NP Variants Loaded with Different Ratios of CpG/VISTA siRNA

For different applications, the optimal ratio of siRNA and CpG may be different to achieve optimal levels of VISTA silencing and simultaneously maximal activation of myeloid cells. As such, we are able to fabricate variants of the immuno-NP with different ratios of VISTA-targeting siRNA to CpG (e.g., siRNA/CpG=5, 3, and 1).

### Deposition and Uptake of Immuno-NPs by Myeloid Cells in TME

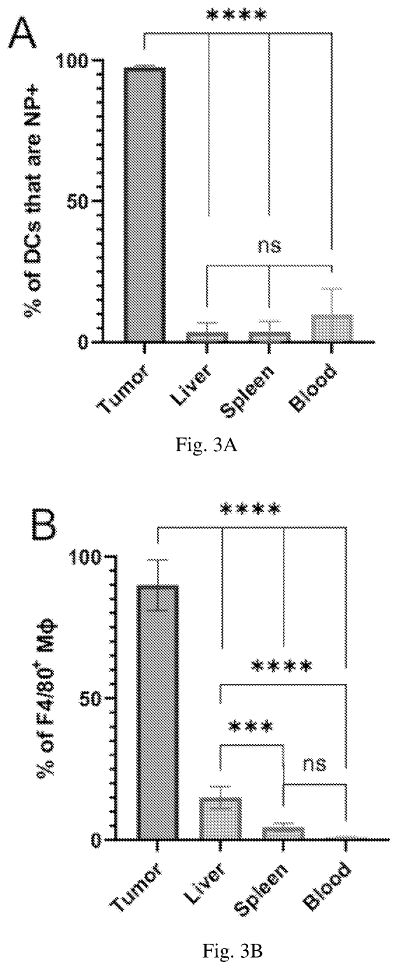

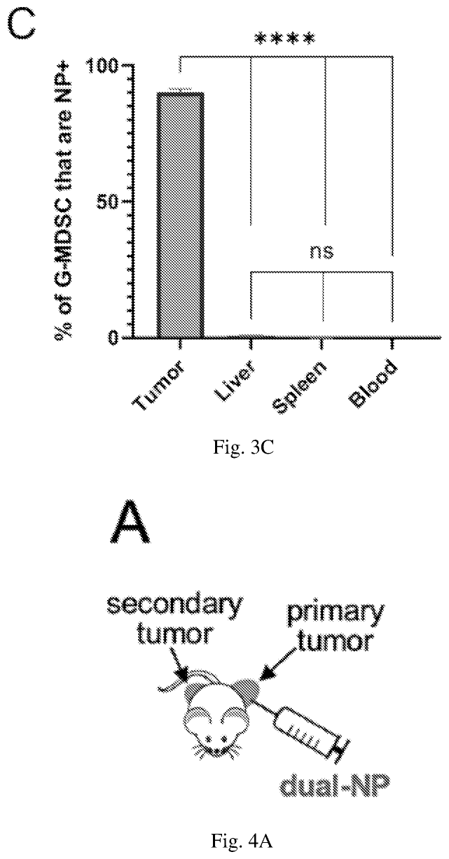

We assessed the cellular uptake in various mouse models of aggressive cancers including melanoma and breast cancer (i.e., B16, YUMM, 4T1, D2A1). We present a subset of these studies in FIG. 3. First, the TME of aggressive tumors is dominated by myeloid cells with a population that is often greater than that of the tumor cells. Second, VISTA is highly expressed on the majority of tumor-associated myeloid cell lineages including MDSCs, TAMs, and DCs. FIG. 3 shows that immuno-NP was found in the majority of tumor-resident myeloid cells.

### Therapeutic Efficacy and Antitumor Immunologic Memory

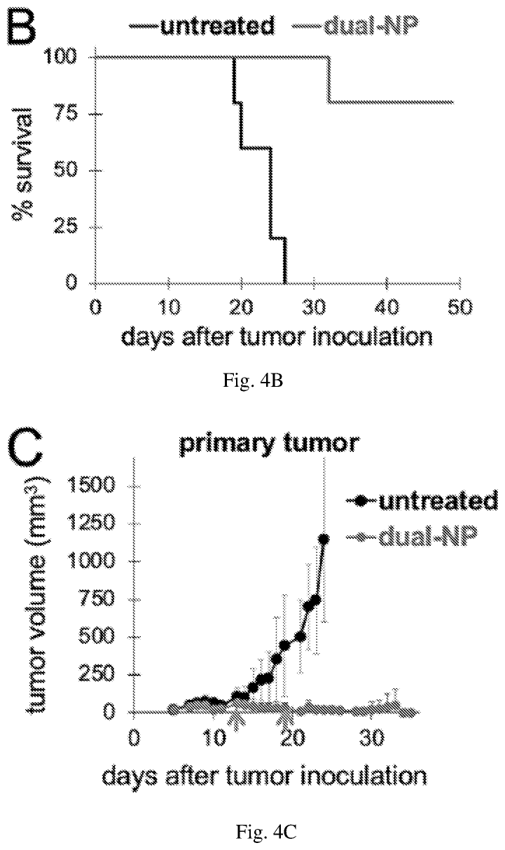

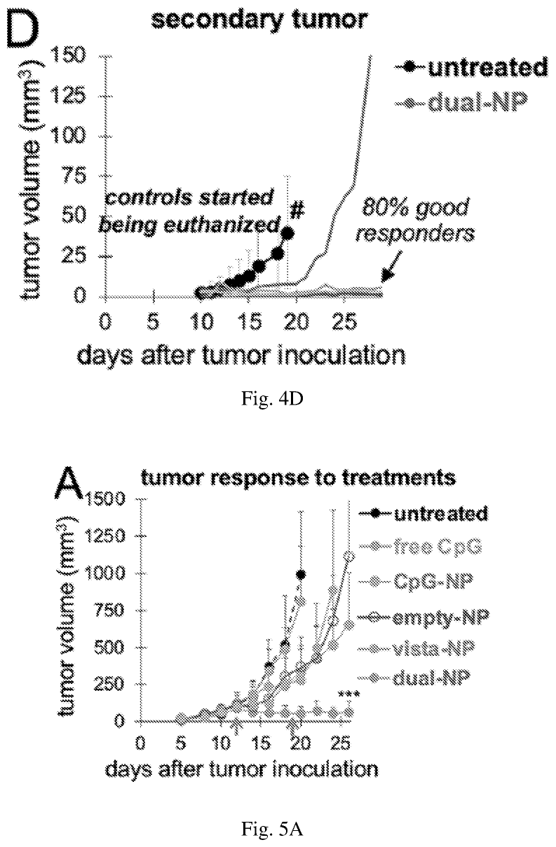

The immuno-NP treatment was tested in various melanoma models. Here, we primarily present data in the B16F10 model, which is the clonal B16 variant with the most malignant and vicious nature. We examined the global effect of the immuno-NP treatment in animals that had two tumors at the same time, a ‘primary’ tumor and a ‘secondary’ smaller tumor (FIG. 4A). The immuno-NP treatment was administered intratumorally (i.t.) only to the ‘primary’ tumor. The benefit to the overall survival of the group treated with immuno-NP was significant (FIG. 4B). Importantly, while the growth of the primary tumor treated with immuno-NP was completely inhibited (FIG. 4C), the ‘secondary’ tumor was also contained in 80% of the group (FIG. 4D). These data indicate that local administration of immuno-NP can have a rapid systemic effect against secondary tumors throughout the body.

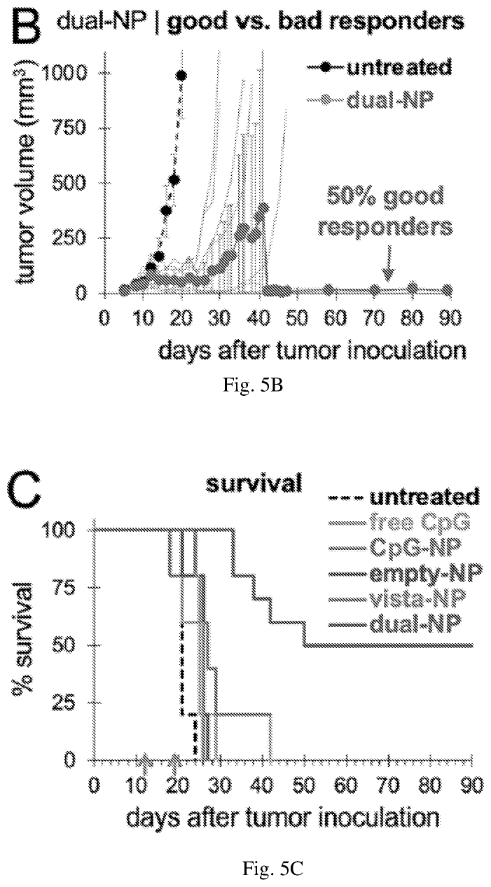

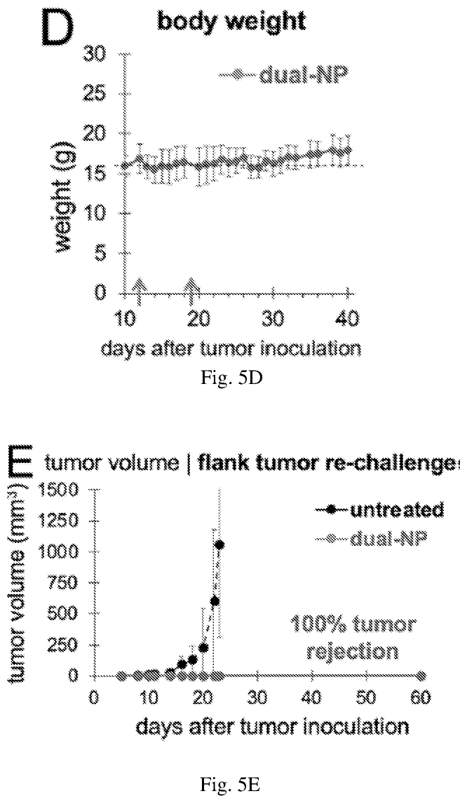

The immuno-NP was compared to control treatments. The administered dose was lower (67%) than the one used in the previous animal study. The immuno-NP significantly outperformed the single-cargo NP variants (CpG-NP and VISTA siRNA-NP), empty NP, and free CpG in terms of tumor growth (FIG. 5A). While i.t. administration of free CpG provided no therapeutic benefits, i.t. delivery of immuno-NP resulted in significant reductions in tumor burden. While mice in the untreated group did not survive for more than 24 days, complete response was achieved in 50% of the immuno-NP-treated mice with these mice being tumor-free for at least 3 months (FIG. 5C). It should be emphasized that this outstanding therapeutic outcome was achieved using just two weekly doses of immuno-NP starting at a point of fully established and rapidly growing tumors. Analysis of body weight post-treatment with immuno-NP indicated no losses in body weight (FIG. 5D). When we challenged the good responders 3 months after the initial tumor inoculation, the flank tumors were rejected in 100% of the group (FIG. 5E, F). The same animals were re-challenged again 2 months later as a B16F10 intravenous model of lung metastatic melanoma. Metastatic disease was rejected in 100% of the immuno-NP-treated mice (FIG. 5G). These results indicate long-lasting antitumor immune protection.

### Effective Targeting of VISTA In Vitro Using Immuno-NP

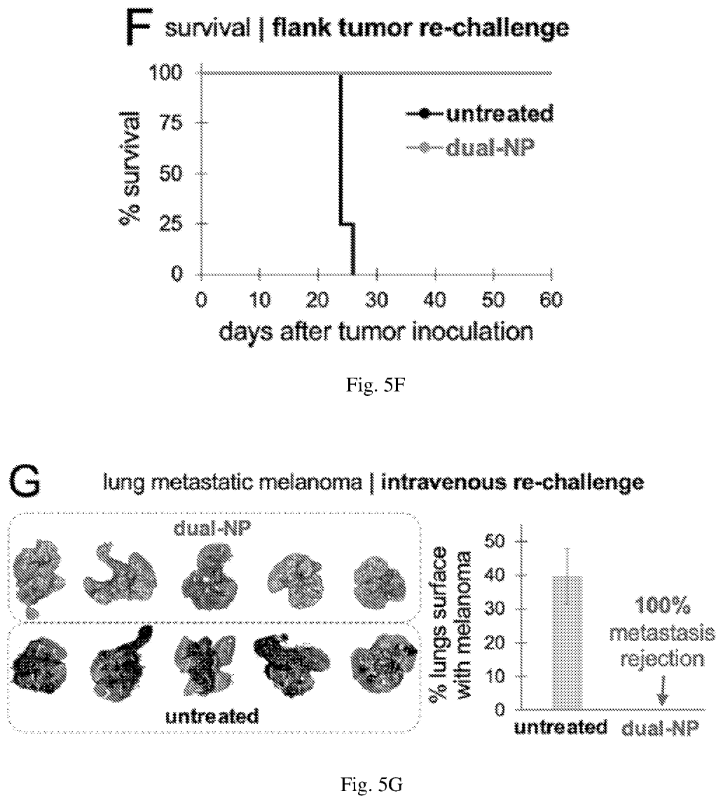

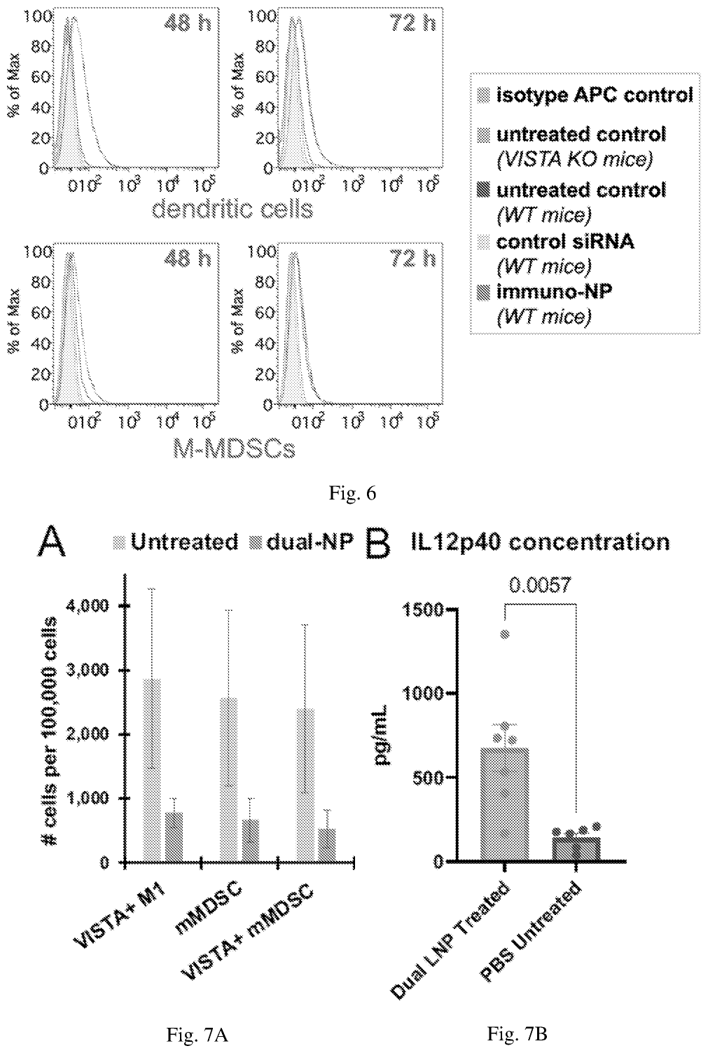

We examined expression of VISTA in freshly isolated DCs and MDSCs from bone marrow of WT and VISTA KO mice. Treatment of the cells with immuno-NP demonstrated that silencing of the VISTA gene resulted in a nearly complete inhibition of VISTA expression in DCs that was sustained for at least 3 days (FIG. 6; top panels). Equally impressive was VISTA targeting in M-MDSCs that was sustained for 2 days (FIG. 6; bottom panels).

### Cellular and Molecular Immune Response to Immuno-NP

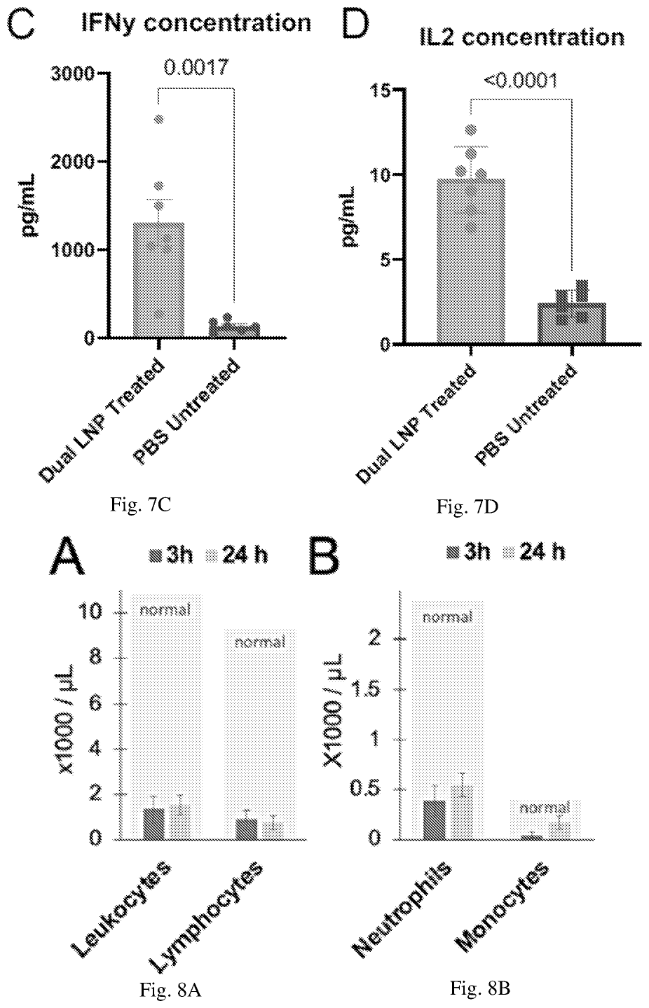

Animals were treated with immuno-NP and tumors were collected and analyzed for immune cell content and VISTA expression (flow cytometry) and key cytokines (ELISA). The immuno-NP reprogrammed the tumor's innate immunity, triggered a significant expansion of proinflammatory innate immune cells and caused a drastic decrease of the dysfunctional and immunosuppressive myeloid cells. It is shown that i.t. administration of the immuno-NP treatment resulted in a significant decrease of VISTA expression in M1 TAMs and MDSCs and an overall significant decrease of the highly immunosuppressive MDSC population (FIG. 7A). Equally important, the immuno-NP treatment triggered a significant production of proinflammatory cytokines (FIG. 7B-D). In summary, these findings strongly support that the immuno-NP enables highly effective VISTA inhibition and synergizes with the TLR9 activation to reprogram the immunosuppressive myeloid cells in the TME.

### Safety Studies

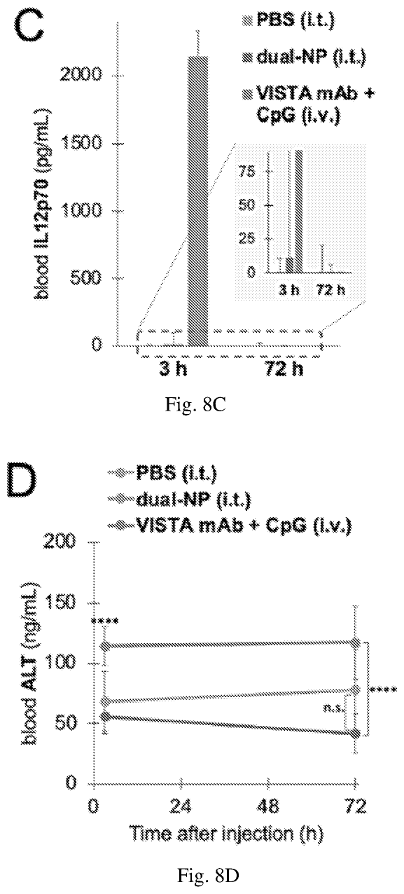

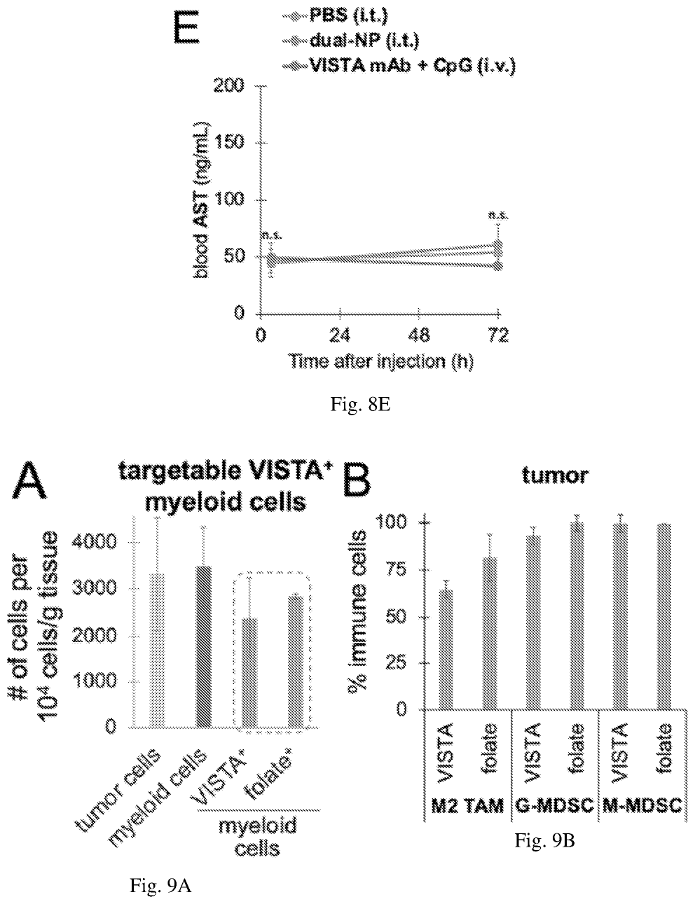

Since safety considerations are paramount for immunotherapies, we performed blood tests to evaluate the risk of acute toxicity and the potential for cytokine storm after i.t. administration of immuno-NP in the B16F10 mouse model. Complete blood count analysis showed that all leukocytes and lymphocytes remained at low levels in the first 24 h after treatment (FIG. 8A). Blood neutrophils and monocytes remained within the normal range (FIG. 8B). In terms of cytokine storm, ELISA analysis showed that immuno-NP resulted in negligible increase of IL12 levels in the blood, while i.v. injection of the standard treatment of VISTA mAb and CpG caused alarmingly high cytokine levels within 3 h after injection (FIG. 8C). Also, we assayed serum levels of alanine aminotransferase (ALT, FIG. 8D) and aspartate aminotransferase (AST, FIG. 8E) as a metric of hepatotoxicity. While both AST and ALT remained at comparable levels to the PBS control, the VISTA mAb and CpG caused signification elevation of ALT. From this preliminary toxicity study, we can conclude that any toxicity associated with immuno-NPs was minimal.

### Targeting Immuno-NPs to Myeloid Cells in TME

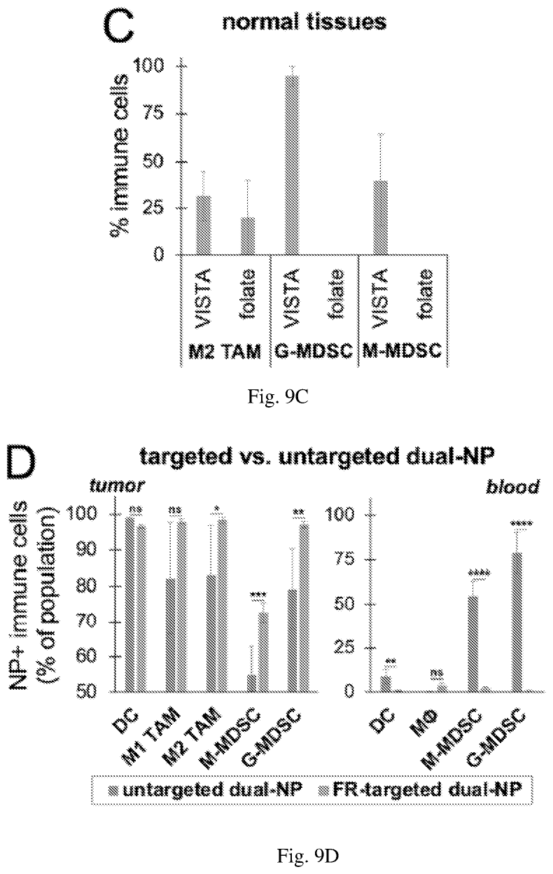

We have significant experience in targeting nanoparticles to aggressive and metastatic tumors. In the case of immuno-NP, it is important to increase targeting of the immuno-NP to the immunosuppressive myeloid cells in TME. Among several suitable candidates, we recently identified a highly attractive target that is specifically overexpressed by the majority of tumor-resident dysfunctional VISTA+myeloid cells in various mouse models of aggressive cancers including melanoma and breast cancer (i.e., B16, YUMM, 4T1, D2A1). We present a subset of these studies in FIG. 9. First, the TME of aggressive melanoma and breast cancer is dominated by myeloid cells with a population that is often greater than that of the tumor cells (FIG. 9A). Second, VISTA is highly expressed on the majority of tumor-associated myeloid cell lineages including MDSCs, TAMs, and DCs. FIG. 9A also shows that the VISTA⁺ myeloid cells were exactly the myeloid cells with high overexpression of folate receptor β (FRβ). Equally important, overexpression of VISTA and FRβ coincides in the majority of G-MDSC, M-MDSC, M2 TAMs and DCs in the TME (FIG. 9B). It is important to emphasize that myeloid cells in healthy tissues have negligible levels of FRβ (FIG. 9C).

As expected, intratumoral administration of FRβ-targeted immuno-NPs resulted in predominant cellular uptake by the majority of MDSCs, and TAMs, which was significantly higher than the untargeted immuno-NP variant (FIG. 9D, left panel). Besides FR-targeting increasing the uptake by myeloid cells in the tumor, targeting also contains the immuno-NP within the tumor boundaries decreasing its ‘spill’ into blood circulation and subsequent uptake by immune cells in the blood (FIG. 9D, right panel), lowering the likelihood of systemic toxicity.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1. An immunostimulatory nanoparticle comprising:
a biocompatible lipid shell that defines an outer surface of the nanoparticle and a core, which is loaded with a Toll-like Receptor 9 (TLR9) agonist and a nucleic acid inhibitor of V domain Immunoglobulin Suppressor of T cell activation (VISTA), and optionally a plurality of targeting moieties linked to the shell and extending from the outer surface, wherein the optional targeting moieties are configured to direct the nanoparticle to tumor-resident myeloid cells in a tumor microenvironment upon administration of the nanoparticle to a subject with cancer.

2. The nanoparticle of claim 1, wherein the TLR9 agonist is a CpG oligonucleotide.

3. The nanoparticle of claim 1, wherein the nucleic acid inhibitor of VISTA includes an RNAi construct that inhibits VISTA expression.

4. The nanoparticle of claim 3, wherein the RNAi construct includes an siRNA targeting VISTA expression.

5. The nanoparticle of claim 1, wherein the TLR9 agonist is a CpG oligonucleotide and the nucleic acid inhibitor of VISTA is a VISTA siRNA.

6. The nanoparticle of claim 5, wherein the ratio of siRNA/CpG loaded into the core is about 10:1 to about 1:10.

7. The nanoparticle of claim 1, wherein the shell is configured to shield the TLR9 agonist and the nucleic acid inhibitor of VISTA from degradation upon administration to the subject and release the TLR9 agonist and the nucleic acid inhibitor of VISTA upon internalization of the nanoparticle by a myeloid cell.

8. The nanoparticle of claim 1, wherein the shell includes at least one ionizable cationic lipid or phospholipid and optionally cholesterol.

9. The nanoparticle of claim 1, wherein the shell includes about 42 mol % to about 50 mol % cationic lipid, about 38 mol % to about 40 mol % cholesterol, and about 11 mol % to about 14 mol % phospholipid.

10. The nanoparticle of claim 1, having a diameter of about 20 nm to about 1 μm.

11. The nanoparticle of claim 1, wherein the targeting moiety includes a ligand that specifically binds to folate receptor beta on myeloid cells.

12. The nanoparticle of claim 1, wherein the ligand comprises folate that is linked to the shell with a PEG linker.

13. An immunotherapy composition comprising:
a plurality of immunotherapy nanoparticles of claim 1 and a pharmaceutically acceptable carrier.

14. (canceled)

15. A method of treating cancer in a subject in need thereof, the method comprising administering to the subject a therapeutically effective amount of the composition of claim 13.

16. The method of claim 15, wherein the administration is selected from systemic administration, intra-tumoral, peri-tumoral, and directly into tumor draining lymph node(s).

17. The method of claim 15, wherein the therapeutically effective amount is the amount effective to reprogram and activate local tumor-resident myeloid cells into T cell-stimulatory cells in the subject and promote cytotoxic CD8+ T-cell mediated killing of tumor cells.

18. The method of claim 15, wherein the therapeutically effective amount is an amount effective to reduce tumor burden in a subject.

19. The method of claim 15, further comprising administering one or more additional cancer therapies to the subject.

20. The method of claim 19, wherein the one or more additional cancer therapies includes radiation therapy, surgery, chemotherapy, an immunotherapy.

21. The method of claim 20, wherein the immunotherapy includes one or more therapeutic antibodies and/or one or more immune checkpoint inhibitors.

22. (canceled)

23. (canceled)
